(19) United States
(12) Patent Application Publication
(10) Pub. No.: US 2026/0274959 A1
(43) Pub. Date: Sep. 17, 2026

(54) ANTIBODY SPECIFICALLY TARGETING B7-H4, AND PREPARATION METHOD THEREFOR AND USE THEREOF

(71) Applicant: FUJIAN MEDICAL UNIVERSITY, Fuzhou (CN)

(72) Inventors: Qiuyu ZHANG, Fuzhou (CN); Renlu ZHANG, Fuzhou (CN); Yang WANG, Fuzhou (CN); Linlin ZHOU, Fuzhou (CN); Yanyang ZHU, Fuzhou (CN)

(21) Appl. No.: 19/162,670

(22) PCT Filed: Jun. 6, 2023
(86) PCT No.: PCT/CN2023/098624

§ 371 Date: Sep. 5, 2025

(30) Foreign Application Priority Data
Apr. 7, 2023 (CN) 202310366940.1

(51) Int. Cl. C07K 16/28; A61K 39/00; A61P 35/00; C12N 15/85

(52) U.S. Cl. CPC C07K 16/2827; A61P 35/00; C12N 15/85; A61K 2039/505; C07K 2317/14; C07K 2317/52; C07K 2317/565; C07K 2317/76

(57) ABSTRACT

The present invention relates to an antibody specifically targeting B7-H4, and a preparation method therefor and the use thereof. In the present invention, the method comprises screening, on the basis of a phage display technique, an antibody variable region targeting B7-H4, then transforming same into a full-length antibody, constructing an expression vector, performing eukaryotic expression and purification so as to obtain the antibody specifically targeting B7-H4.

### RELATED APPLICATIONS

This application claims priority to Chinese patent application No. 202310366940.1, entitled “ANTIBODY SPECIFICALLY TARGETING B7-H4, AND PREPARATION METHOD THEREFOR AND USE THEREOF” filed on Apr. 7, 2023, the content of which is hereby incorporated by reference in its entirety.

### TECHNICAL FIELD

The present disclosure relates to the field of biomedicine, in particular to an antibody specifically targeting B7 Homolog 4 (B7-H4), a preparation method thereof, and use thereof.

### BACKGROUND

Malignant tumors have become the primary factor threatening human health and reducing life expectancy. Tens of millions of new cancer patients are diagnosed every year. Conventional tumor treatment methods such as chemotherapy, radiotherapy, and surgery have limited effects and large toxic side effects, which have been changed by the establishment and vigorous development of tumor immunotherapies. After remarkable results in tumor treatment were achieved by blocking cytotoxic T lymphocyte-associated antigen-4 (CTLA-4) and programmed cell death-1 (PD-1) as two firstly detected immune checkpoints, a new wave of exploration of tumor treatment methods based on blocking immune checkpoint ligand and receptor has emerged. Compared to conventional treatment methods, tumor therapeutic antibodies targeting immune regulation can selectively block specific immunosuppressive pathways to indirectly kill tumor cells, reduce damage to normal tissues, are less likely to develop drug resistance, and have good safety and tolerability. So far, the Food and Drug Administration (FDA) has approved nearly 50 monoclonal antibody drugs for tumor treatment, but the limited number of patients who have been benefited is still a prominent problem in current tumor treatment. Therefore, in-depth research on the regulatory mechanisms of tumor immunity and exploration of other targeted drugs and inhibitory pathways become the focus of the tumor immunity field.

In the past few years, more and more studies have shown that B7-H4 is a potential target for tumor treatment. The B7 family molecules are a group of transmembrane proteins that are involved in stimulating or inhibiting T cell immune response-related signals. B7-H4 is a member of the B7 family, and its homology with DNA sequences of other molecules of the B7 family was identified by three laboratories in 2003. In healthy individuals, relatively low mRNA levels of B7-H4 are detected in many non-lymphoid tissues such as placenta, lung, liver, ovary, and spleen. However, abnormally high expression of B7-H4 can be observed in various types of tumor tissues, such as breast cancer, gastric cancer, colorectal cancer, lung cancer, prostate cancer, melanoma, and renal cell carcinoma. The expression level of B7-H4 is negatively correlated with prognosis and overall survival of patients. Among the molecules of the B7 family, B7-H4 has the smallest overlap in co-expression with programmed cell death ligand 1 (PD-L1). Chen D et al. analyzed the mRNA expression levels of PD-L1 and B7-H4 based on two published RNA-seq datasets of glioma patients obtained from TCGA and CGGA, and observed a significant negative correlation between PD-L1 and B7-H4. Song X et al. detected the protein expression levels of PD-L1 and B7-H4 in 45 breast cell lines and 4 ovarian cell lines, in which B7-H4 was detected in 60.9% (25/41) of the tested breast cancer cell lines (10 of which were TNBC cell lines), while PD-L2 and PD-L1 were respectively expressed in 10% and 25% of the tested cell lines. The immunohistochemistry (IHC) array performed on a tissue array of 110 breast infiltrating ductal carcinomas also confirmed that the expression of B7-H4 was negatively correlated to the expression of PD-L1. Similar results were also observed in tumors such as lung adenocarcinoma, lung cancer, cervical cancer, etc. Thus, B7-H4 is expected to become an immune checkpoint molecule that compensates for PD-L1 to exert an immunosuppressive effect in the tumor microenvironment of patients with low PD-L1 expression or resistance to PD-L1 antibody therapy. In addition, B7-H4 is highly expressed in breast cancer cells and tends to increase its expression level in triple-negative breast cancer cells. Therefore, B7-H4 has the potential to be a new target for the treatment of primary and recurrent breast cancer, including triple-negative breast cancer that is ineffective for HER2 targeted therapy. It has been reported that B7-H4 mediates tumor immune escape by inhibiting T cell responses. Previous studies of our group have shown that B7-H4 expressed by tumor cells can exert immunosuppressive effects by inhibiting activation, proliferation and killing ability in vivo of CD8 T cells, suggesting that B7-H4 expressed by tumor cells may also be an immunotherapy target based on T cell activation strategy. B7-H4 can further enhance the proliferation, invasion, metastasis, and anti-apoptosis of tumor cells by participating in cell signal transduction pathways, thereby promoting the development of tumor. Therefore, if the function of the B7-H4 protein is inhibited, its contact with B7-H4 receptors or ligands on T cells may be blocked, which affects signal transduction, thereby inhibiting the proliferation or escape of tumor cells.

Although B7-H4 has a great potential as an anti-tumor immune target, there is no B7-H4 targeting antibody drug on the market so far, nor is there fully human neutralizing antibody against natural B7-H4. There have been some reports on the in vitro and in vivo anti-tumor activity of antibodies targeting B7-H4. For example, Prasad DVR et al. showed that in the presence of B7-H4 antibody (clone 54), IL-2 secretion is increased, and proliferation and activation levels of T cells are increased. The anti-mouse B7-H4 monoclonal antibodies (8H4 and 8H4.1) developed by Sica G L et al. exhibited the effect of weakening B7-H4 positive tumor cells. It has also been reported that the anti-mouse B7-H4 antibody (3E8) can reverse the inhibitory effect of B7-H4 protein on the release of cytokines by activated mouse T cells. In addition, the anti-mouse B7-H4 antibody can increase the anti-tumor effect of T cells in tumor-bearing mice and inhibit the growth of B7-H4 positive mouse lung cancer model in vivo. Regarding the antibodies against human B7-H4, Xue Q et al. discovered that an anti-human B7-H4 antibody (3C8) can significantly reduce the inhibitory effect of B7-H4 expressed by human bone marrow-derived mesenchymal stem cells on T cells, but no anti-tumor efficacy was observed in a xenograft model. Due to the weak binding of mouse monoclonal antibodies to human Fc receptors, a series of indirect protection mechanisms will be lost, such as antibody-dependent cell-mediated cytotoxicity, complement-dependent cytotoxicity, phagocytosis, opsonization, etc. In addition, the half-life of mouse monoclonal antibodies is short, which requires to apply more mouse monoclonal antibodies and multiple administrations during treatment to achieve the desired therapeutic effect. More importantly, mouse antibodies have strong immunogenicity, which limits their clinical applications. Currently, 7 mouse antibodies obtained through hybridoma preparation technology have entered clinical trials. Due to the lack of direct anti-tumor activity, they have been prepared into bispecific antibodies or antibody-drug conjugates (ADCs) with other target antibodies or small molecule drugs by researchers, in which three antibodies were prepared into bispecific antibodies with other target antibodies, i.e., HBM7008 (B7-H4×4-1BB BsAb), GEN-1047 (B7-H4×CD3 BsAb), and PF-07260437 (B7-H4×CD3 BsAb), and four antibodies (i.e., AZD-8205, HS-20089, XMT-1660, and SGN-B7-H4V) were prepared into ADCs by coupling with small molecule drugs. The B7-H4 antibodies (i.e., FPA150 and NC762 antibodies), which have entered clinical phases I and II, were obtained by immunizing humanized mice and B7-H4 knockout mice, respectively. Antibodies derived from mouse immunization cannot overcome murine components, which may trigger the generation of anti-mouse antibodies in human, in turn affecting the efficacies of the antibodies in vivo. Therefore, generating a fully human B7-H4 neutralizing antibody with high specificity can contribute to improving the clinical application of immunotherapy strategies targeting the B7-H4 molecule.

Based on the above, the present disclosure is proposed.

### SUMMARY

On this basis, an antibody specifically targeting B7-H4, a preparation method thereof, and use thereof are provided according to various embodiments of the present disclosure. The technical solutions are proposed as follows.

In a first aspect of the present disclosure, an antibody specifically targeting B7-H4 is provided. The antibody includes a heavy chain CDR1 shown in SEQ ID NO: 6, a heavy chain CDR2 shown in SEQ ID NO: 7, and a heavy chain CDR3 shown in SEQ ID NO: 8; and a light chain CDR1 shown in SEQ ID NO: 9, a light chain CDR2 shown in SEQ ID NO: 10, and a light chain CDR3 shown in SEQ ID NO: 11.

In some embodiments of the present disclosure, the antibody includes a heavy chain variable region shown in SEQ ID NO: 4.

In some embodiments of the present disclosure, the neutralizing antibody includes a light chain variable region shown in SEQ ID NO: 5.

In some embodiments of the present disclosure, the antibody further includes a heavy chain constant region and a light chain constant region.

In some embodiments of the present disclosure, the heavy chain constant region and the light chain constant region are each independently derived from any species selected from the group consisting of human, mouse, rabbit, sheep, cow, horse, pig, dog, cat, camel, donkey, deer, mink, chicken, duck, and goose.

In some embodiments of the present disclosure, the heavy chain constant region and the light chain constant region each independently include a constant region sequence derived from any one of IgG1, IgG2, IgG3, IgG4, IgA, IgM, IgE, or IgD.

In a second aspect of the present disclosure, a recombinant protein is provided. The recombinant protein includes one or more of the heavy chain CDR1, the heavy chain CDR2, the heavy chain CDR3, the light chain CDR1, the light chain CDR2, the light chain CDR3, the heavy chain variable region, and the light chain variable region defined in the first aspect.

In a third aspect of the present disclosure, a detection reagent, a detection kit, or a medicament are provided. The detection reagent, the detection kit, or the medicament include the antibody in the first aspect or the recombinant protein in the second aspect.

In a fourth aspect of the present disclosure, a nucleic acid is provided. The nucleic acid includes a nucleic acid fragment for encoding the antibody in the first aspect or the recombinant protein in the second aspect.

In a fifth aspect of the present disclosure, a recombinant expression vector is provided. The recombinant expression vector includes the nucleic acid in the fourth aspect.

In some embodiments of the present disclosure, the recombinant expression vector is an antibody expression vector.

In some embodiments of the present disclosure, the recombinant expression vector is selected from the group consisting of a bacterial plasmid, a bacteriophage, a yeast plasmid, a plant cell virus, a mammalian cell virus, and a combination thereof.

In some embodiments of the present disclosure, the recombinant expression vector is a PCDNA3.1 expression vector.

In a sixth aspect of the present disclosure, a host cell is provided. The host cell includes the nucleic acid in the fourth aspect, or the recombinant expression vector in any embodiments of the fifth aspect.

In some embodiments of the present disclosure, the host cell is a CHO cell, a COS cell, an NSO cell, a HeLa cell, a BHK cell, or a HEK293 cell.

In a seventh aspect of the present disclosure, a method for preparing the antibody in the first aspect or the recombinant protein in the second aspect is provided. The preparation method includes the following steps: culturing the host cell in the sixth aspect, and isolating the antibody or the recombinant protein from the obtained culture.

In an eighth aspect of the present disclosure, provided is use of the antibody in the first aspect or the recombinant protein in the second aspect in the manufacture of a medicament for treating tumors.

In some embodiments of the present disclosure, the tumor is a colorectal cancer that is positive for B7-H4 expression.

The details of one or more embodiments of the present disclosure are set forth in the description below, and other features, objectives, and advantages of the present disclosure will be apparent from the description and claims.

### BRIEF DESCRIPTION OF THE DRAWINGS

In order to illustrate the technical solutions in the embodiments of the present disclosure or in the conventional technologies more clearly, the drawings used in describing the embodiments or the conventional technologies will be described briefly. Apparently, the following described drawings are merely for the embodiments of the present disclosure, and other drawings can be derived based on these drawings by those of ordinary skill in the art without any creative effort.

FIG. 1 shows the result of construction of a phage display human natural single-chain antibody library in Example 1.

FIG. 2 shows positive monoclonal screening results in Example 3.

FIG. 3 shows graphs of specificity and affinity results of single-chain antibody candidates in Example 4.

FIG. 4 is an image showing the construction result of an expression vector expressing the Npsc-A8 full-length antibody in Example 5.

FIG. 5 shows images of expression and purification results of the Npsc-A8 full-length antibody in Example 5.

FIG. 6 shows graphs of specificity comparison results of the Npsc-A8 full-length antibody in Example 6.

FIG. 7 shows graphs of binding site analysis results of the Npsc-A8 full-length antibody in Example 7.

FIG. 8 shows graphs of results of the Npsc-A8 full-length antibody reversing B7-H4-mediated suppression of T cell function and promoting T cell proliferation in Example 8.

FIG. 9 shows graphs of results of the Npsc-A8 full-length antibody promoting T cells to kill B7-H4 positive tumor cells in Example 9.

FIG. 10 shows graphs of therapeutic efficacy of anti-B7-H4 blocking antibody in Example 10.

### DETAILED DESCRIPTION

The technical solutions in embodiments of the present disclosure will now be described clearly and completely with reference to the accompanying drawings in embodiments of the present disclosure. Obviously, the described embodiments are only part of, but not all of the embodiments of the present disclosure. Based on the embodiments in the present disclosure, other embodiments obtained by those skilled in the art without creative work are all within the scope of protection of present disclosure.

Unless otherwise defined, all technical and scientific terms used herein have the same meaning as commonly understood by those skilled in the art to which the present disclosure belongs. The terms used herein in the specification of the present disclosure are used only for the purpose of describing embodiments and are not intended to limit the present disclosure.

### Terminology

Unless otherwise stated or there is a contradiction, terms or phrases used herein have the following meanings.

The terms “and/or”, “or/and”, and “as well as/or” used herein include both any one of two or more related listed items, and any or all combinations of related listed items. The any or all combinations include combinations of any two related listed items, combinations of any multiple related listed items, or a combination of all related listed items. It should be noted that, when at least three items are connected by at least two conjunctions of “and/or”, “or/and”, and “as well as/or”, it should be understood that, in the present disclosure, the technical solutions undoubtedly include not only technical solutions that are all connected by “logical AND”, but also technical solutions that are all connected by “logical or”. For example, “A and/or B” includes three parallel solutions: A alone, B alone, and a combination of A and B. For example, the technical solution of “A, and/or, B, and/or, C, and/or, D” includes any one of A, B, C, and D (that is, the technical solutions that are connected by “logical OR”), any or all combinations of A, B, C, and D (that is, combinations of any two or any three of A, B, C, and D), and the combination of four of A, B, C, and D (that is, the technical solution that is connected by “logical AND”).

In the present disclosure, “plurality”, “multiple”, “multiple times”, “multiples”, etc., unless otherwise specified, refer to a number greater than 2 or equal to 2. For example, “one or more” means one or greater than or equal to two.

Herein, “preferred”, “better”, “more preferred” and “appropriate” are merely used to describe embodiments or examples with better effects. It should be understood that they do not constitute limitations on the scope of protection of present disclosure.

In the present disclosure, the technical features described in an open-type manner include both closed-ended technical solutions of the listed features, and open-ended technical solutions of the listed features.

In the present disclosure, when referring to a numerical range, unless otherwise specified, the distribution of optional numerical values within the numerical interval is deemed to be continuous and includes the two numerical endpoints of the numerical range (i.e., the minimum value and the maximum value), as well as each numerical value between the two numerical endpoints. Unless otherwise specified, when a numerical interval refers only to integers within the numerical interval, it includes the two endpoint integers of the numerical range and every integer between the two endpoints, which is equivalent to directly listing every integer. For example, t is an integer selected from 1 to 10, which indicates that t is any integer selected from the integer group consisting of 1, 2, 3, 4, 5, 6, 7, 8, 9, and 10. In addition, when multiple ranges are provided to describe features or characteristics, these ranges can be combined. In other words, unless otherwise specified, the ranges disclosed herein should be understood to include any and all sub-ranges included therein.

The temperature parameter in present disclosure, unless otherwise specified, is allowed to be either constant temperature treatment, or allowed to vary within a certain temperature range. It should be understood that the constant temperature treatment allows fluctuation of the temperature within an accuracy range controlled by the instrument, and fluctuations within the range of ±5° C., ±4° C., ±3° C., ±2° C., or ±1° C. are allowed.

In the present disclosure, “% (w/w)” and “wt %” both represent weight percentage, % (v/v) refers to volume percentage, and % (w/v) refers to mass volume percentage.

All documents mentioned in the present disclosure are incorporated by reference into present disclosure to the same extent as if each individual document was individually incorporated by reference. Unless they conflict with the invention purpose and/or technical solution of present disclosure, the cited documents involved in the present disclosure are cited with all contents and for all purposes. When the present disclosure involves cited documents, the definitions of relevant technical features, terms, nouns, phrases, and the like in the cited documents are also cited. When the present disclosure involves the cited documents, the cited examples and preferred ways of the relevant technical features may also be incorporated into the present disclosure as references, but only to the extent that the present disclosure can be implemented. It should be understood that when the cited contents conflict with the description in the present disclosure, the present disclosure shall prevail or adaptive amendments shall be made according to the description in the present disclosure.

As used herein, the term “B7-H4” refers to B7-H4 polypeptides in mammalian, including but being not limited to native B7-H4 polypeptides and isoforms of B7-H4 polypeptides. “B7-H4” encompasses full-length B7-H4 polypeptides, unprocessed B7-H4 polypeptides, as well as B7-H4 polypeptides produced by processing in cells. As used herein, the term “human B7-H4” refers to a polypeptide including the amino acid sequence described above. “B7-H4 polynucleotide”, “B7-H4 nucleotide”, or “B7-H4 nucleic acid” all refers to a polynucleotide encoding B7-H4.

The term “antibody” means an immunoglobulin molecule that recognizes and specifically binds to a target (such as a protein, a polypeptide, a peptide, carbohydrate, a polynucleotide, a lipid, or a combination thereof) by at least one antigen recognition site located in the variable region of the immunoglobulin molecule. As used herein, the term “antibody” encompasses complete polyclonal antibodies, complete monoclonal antibodies, chimeric antibodies, humanized antibodies, human antibodies, fusion proteins including antibodies, and any other modified immunoglobulin molecules, as long as the antibodies exhibit the desired biological activity. The antibody can belong to any of the following five major classes of immunoglobulins: IgA, IgD, IgE, IgG and IgM, or their subclasses (i.e., isotypes), e.g., IgG1, IgG2, IgG3, IgG4, IgAQ1 and IgA2, and they are referred to as a, 8, &, Y or μ chains respectively, based on the properties of their heavy chain constant structures. Different types of immunoglobulins have different and well-known subunit structures and three-dimensional configurations. Antibodies can be naked or conjugated with other molecules such as toxins, radioisotopes, etc.

The term “antibody fragment” refers to a portion of an intact antibody. “Antigen binding fragment”, “antigen binding domain” or “antigen binding region” refers to a portion of an intact antibody that specifically binds to an antigen. An antigen binding fragment can contain an antigen recognition site an intact antibody, e.g., a complementarity-determining region (i.e., CDR region) sufficient to specifically bind to an antigen. Examples of antigen binding fragments of antibodies include, but are not limited to, Fab, Fab′, F(ab′)2, and Fv fragments, linear antibodies, and single-chain antibodies. Antigen binding fragments of antibodies can be derived from any animal species, such as rodents (e.g., mice, rats, or hamsters) and humans, or can be artificially produced.

The terms “anti-B7-H4 antibody”, “B7-H4 antibody”, and “antibody that binds to B7-H4” all refer to antibodies that are capable of specifically binding to B7-H4 with sufficient affinity, so that the antibodies can be used as diagnostic and/or therapeutic agents in targeting B7-H4. As used herein, the terms “specifically binds”, “immunospecifically binds”, “immunospecifically recognizes”, and “specifically recognizes” are analogous terms in the context of an antibody or antigen-binding fragment thereof, which indicate that the antibody or antigen-binding fragment thereof binds to an epitope via its antigen-binding domain, and that binding requires a certain complementarity between the antigen-binding domain and the epitope. Thus, an antibody that “specifically binds” to human B7-H4 shown in SEQ ID NO: 1 can also bind to B7-H4 derived from other species (e.g., B7-H4 derived from cynomolgus monkey, mouse, and/or rat), and/or B7-H4 proteins produced by other human alleles. However, the extent of binding of the anti-B7-H4 antibody to unrelated, non-B7-H4 proteins (e.g., other B7 protein family members, such as PD-L1) is less than about 10% of the extent of binding of the antibody to B7-H4, as measured, for example, by radioimmunoassay.

A “monoclonal” antibody or antigen-binding fragment thereof refers to a homogeneous population of antibodies or antigen-binding fragments that participate in highly specific recognition and binding of a single antigenic determinant or a single epitope. The monoclonal antibody contrasts with polyclonal antibodies which typically include different antibodies targeting different antigenic determinants. The term “monoclonal antibody” or antigen-binding fragment thereof encompasses intact and full-length monoclonal antibodies as well as antibody fragments such as Fab, Fab′, (Fab′)2, Fv, scFv mutants, fusion proteins including the antibody, and any other modified immunoglobulin molecules including an antigen recognition site. In addition, a “monoclonal” antibody or antigen-binding fragment thereof refers to such antibodies and antigen-binding fragments thereof prepared in any number of methods, including but being not limited to hybridomas, phage selection, recombinant expression, and transgenic animals.

As used herein, the terms “variable region” or “variable domain” are used interchangeably and are common in the art. The variable region generally refers to a portion of an antibody, generally a portion of a light chain or a portion of a heavy chain, usually about 110 to 120 amino acids or 110 to 125 amino acids at the amino-terminal in a mature heavy chain, or about 90 to 115 amino acids at the amino-terminal in a mature light chain, which differ in sequence between antibodies and are used for the binding and specificity between a particular antibody and its specific antigen. The variability of the sequence is concentrated in CDR regions, while the more conserved regions in the variable region are called framework regions (i.e., FR regions). Without wishing to be bound by any particular mechanism or theory, it is believed that the CDRs of the light and heavy chains are primarily responsible for the interaction and specificity of the antibody with the antigen. In some embodiments, the variable region is a human variable region. In some embodiments, the variable region is a variable region derived from rodents or murine.

The terms “VL” and “VL domain” are used interchangeably to refer to a light chain variable region of an antibody.

The terms “VH” and “VH domain” are used interchangeably to refer to a heavy chain variable region of an antibody.

The term “Kabat numbering” and similar terms are well-known in the art and refer to a system for numbering amino acid residues in the heavy and light chain variable regions of an antibody or antigen-binding fragment thereof. In certain aspects, CDRs can be determined according to the Kabat numbering system, see, e.g., Kabat EA and Wu TT (1971) Ann NY Acad Sci 190:382-391, and Kabat E A et al., (1991) Sequences of Proteins of Immunological Interest, Fifth Edition, United States Department of Health and Human Services, NIH Publication No. 91-3242. According to the Kabat numbering system, CDRs in a heavy chain of an antibody molecule are typically located at amino acid positions 31 to 35, which can optionally include one or two additional amino acids after position 35, referred to as 35A and 35B in the Kabat numbering system (CDR1); at amino acid positions 50 to 65 (CDR2); and at amino acid positions 95 to 102 (CDR3). According to the Kabat numbering system, CDRs in a light chain of an antibody molecule are typically located at amino acid positions 24 to 34 (CDR1), at amino acid positions 50 to 56 (CDR2), and at amino acid positions 89 to 97 (CDR3). In a specific embodiment, the CDRs of the antibodies described in the present disclosure have been determined according to the Kabat numbering system.

As used herein, the terms “constant region” and “constant domain” are interchangeable and have their common meanings in the art. The constant region is a portion of an antibody that is not directly involved in binding of the antibody to an antigen, but can exhibit a variety of effector functions, such as interaction with a Fc receptor, for example, a carboxyl terminal portion of a light chain and/or a heavy chain. Relative to the variable domain of the immunoglobulin, the constant region of the immunoglobulin molecule generally has a more conservative amino acid sequence. In some aspects, the antibody or antigen-binding fragment includes a constant region or a portion thereof sufficient for antibody-dependent cell-mediated cytotoxicity (ADCC).

As used herein, based on the amino acid sequence of the constant domain, the term “heavy chain” can refer to any of the different types when used in relation to antibodies, e.g., a, 8, &, Y, and μ to form antibodies of IgA, IgD, IgE, IgG, and IgM categories, respectively, including subclasses of IgG, e.g., IgG1, IgG2, IgG3, and IgG4. Heavy chain amino acid sequences are well known in the art. In a specific embodiment, the heavy chain is a human heavy chain. In a specific embodiment, the heavy chain is a heavy chain derived from rodents or murine.

As used herein, based on the amino acid sequence of the constant domain, the term “light chain” can refer to any of the different types when used in relation to antibodies, such as kappa or lambda. Light chain amino acid sequences are well known in the art. In a specific embodiment, the light chain is a human light chain. In a specific embodiment, the light chain is a light chain derived from rodents or murine.

The term “chimeric” antibody or antigen-binding fragment thereof refers to an antibody or antigen-binding fragment thereof in which the amino acid sequence is derived from two or more species. Typically, the variable regions of the light chain and the heavy chain correspond to the variable regions of an antibody or antigen-binding fragment thereof derived from one mammalian species (e.g., mouse, rat, rabbit, etc.) with the desired specificity, affinity, etc., while the constant regions of the light chain and the heavy chain are homologous to sequences in an antibody or antigen-binding fragment thereof derived from another species, usually being human, to avoid eliciting an immune response in the species.

The term “human” antibody or antigen-binding fragment thereof refers to an antibody or antigen-binding fragment thereof having an amino acid sequence derived from a human immunoglobulin locus, wherein such antibody or antigen-binding fragment thereof is prepared by using any technique known in the art. The human antibody or antigen-binding fragment thereof is defined to include a complete or full-length antibody and a fragment thereof.

“Binding affinity” generally refers to the total strength of non-covalent interaction between a single binding site of a molecule, e.g., an antibody, or antigen-binding fragment thereof, and its binding partner, e.g., an antigen. Unless otherwise indicated, as used herein, “binding affinity” refers to intrinsic binding affinity, which reflects the interaction between members of a binding pair (e.g., an antibody or antigen-binding fragment thereof and an antigen) in a ratio of 1:1. The affinity between a molecule X and its partner Y can generally be represented by a dissociation constant (K). The affinity can be measured and/or represented in a variety of ways known in the art, including, but being not limited to, an equilibrium dissociation constant.

As used herein, “epitope” is a term in the art that refers to a local region of an antigen to which an antibody or antigen-binding fragment thereof can specifically bind. An epitope can be, for example, a continuous amino acid of a polypeptide (i.e., a linear or continuous epitope), or for example, two or more non-continuous regions from one or more polypeptides (i.e., a conformational, non-linear, discontinuous, or non-continuous epitope. In some embodiments, the epitope to which an antibody or antigen-binding fragment thereof specifically binds can be determined by, for example, NMR spectroscopy, X-ray diffraction crystallography studies, ELISA assays, hydrogen/atmosphere exchange in combination with mass spectrometry (e.g., liquid chromatography electrospray mass spectrometry), array-based oligopeptide scanning assays, and/or mutagenesis mapping (e.g., site-directed mutagenesis mapping). For X-ray crystallography, crystallization can be accomplished using any method known in the art (e.g., Giegé R et al. (1994) Acta Crystallogr D Biol Crystallogr 50 (Pt 4): 339-350; McPherson A (1990) Eur J Biochem 189:1-23; Chayen NE (1997) Structure 5:1269-1274; McPherson A (1976) J Biol Chem 251:6300-6303). Antibodies/antigen-binding fragments thereof: Antigen crystals can be studied using well-known X-ray diffraction techniques and can be improved using computer software such as X-PLOR (Yale University, 1992, distributed by Molecular Simulations, Inc.; see, e.g., Meth Enzymol (1985) Vols. 114 and 115, edited by Wyckoff H W et al.; US2004/0014194) and BUSTER (Bricogne G (1993) Acta Crystallogr D Biol Crystallogr 49 (Pt 1): 37-60; Bricogne G (1997) Meth Enzymol 276A: 361-423, edited by Carter CW; Roversi P et al., (2000) Acta Crystallogr D Biol Crystallogr 56 (Pt 10): 1316-1323). Mutagenesis mapping studies can be performed using any method known to those skilled in the art. For a description of mutagenesis techniques, including alanine scanning mutagenesis, see, for example, Champe M et al. (1995) J Biol Chem 270:1388-1394 and Cunningham BC and Wells JA (1989) Science 244:1081-108.

A B7-H4 antibody that “binds to the same epitope” as a reference B7-H4 antibody refers to an antibody that binds to the same B7-H4 amino acid residue as the reference B7-H4 antibody. The ability of the B7-H4 antibody binding to the same epitope as the reference B7-H4 antibody is determined by a hydrogen-deuterium exchange assay, see Coales et al. Rapid Commun. Mass Spectrom. 2009; 23:639-647.

If an antibody binds to a given epitope or an overlapping epitope of a reference antibody such that it blocks the binding of the reference antibody to the epitope to some extent, the antibody can be considered to “competitively inhibit” the binding of the reference antibody to the epitope. Competitive inhibition can be determined by any method known in the art, for example, a competitive ELISA assay. An antibody can competitively inhibit the binding of the reference antibody to a given epitope by at least 90%, at least 80%, at least 70%, at least 60%, or at least 50%.

The term “overexpression” of B7-H4 in a particular tumor, tissue, or cell sample refers to the presence of B7-H4 (i.e., a B7-H4 polypeptide, or a nucleic acid encoding such polypeptide) at a level higher than that of B7-H4 present in cells of the same type or origin or other cells in non-diseased tissue or adjacent to the tumor or cancer. Such overexpression can be caused, for example, by mutation, gene amplification, increased transcription, or increased translation.

The “sample” in the present disclosure has a biological origin. In a preferred embodiment, the sample is a human sample, but animal samples can also be used in the practice of the present disclosure. Non-limiting sources for the sample used in the present disclosure include, for example, solid tissues, biopsies, ascites, aspirates, fluid leaching, blood including circulating tumor cells) plasma, serum, cerebrospinal fluid, lymph, external sections of skin, respiratory tract, intestinal and urogenital tracts, tears, saliva, milk, tumors, organs, cell cultures, and/or cell culture components.

The term “label” when used herein refers to a detectable compound or composition that is conjugated directly or indirectly to the antibody so as to generate a “labeled” antibody. The label can itself be detectable, e.g., radioisotope labels or fluorescent labels. Alternatively, in the case of an enzyme for labeling, the label can catalyze chemical alteration of a substrate compound or composition that is detectable.

Terms such as “treat”, “treatment”, “treating”, “alleviate”, and “relieve” refer to therapeutic measures that can cure, slow down, alleviate the symptoms of a pathological disorder or condition, and/or prevent the progression thereof. Therefore, those in need of treatment include those who have been diagnosed with or suspected of having the disorder. In some embodiments, the cancer in a patient is successfully “treated” according to the method of the present disclosure if the patient shows one or more of the following: a decrease in the number of cancer cells or the complete absence of the cancer cells; a decrease in tumor size; inhibition or absence of infiltration of cancer cells into surrounding organs, including, for example, cancer spread to soft tissue and bone; inhibition or absence of tumor metastasis; inhibition or absence of tumor growth; alleviation of one or more symptoms associated with a particular cancer; reduced morbidity and death rate; improved quality of life; reduced tumorigenicity, tumorigenic frequency, or tumorigenic capacity of a tumor; reduced number or frequency of cancer stem cells in a tumor; differentiation of tumorigenic cells into a non-tumorigenic state; increased progression-free survival (PFS), increased disease-free survival (DFS), increased overall survival (OS), increased complete response (CR), partial response (PR), stable disease (SD), reduced progressive disease (PD), reduced time to progression (TTP), or any combination thereof.

The terms “cancer” and “cancerous” refer to or describe the physiological condition in mammals in which a population of cells is characterized by unregulated cell growth. Examples of cancers include, but are not limited to, gynecological cancers (e.g., breast cancer including triple-negative breast cancer, ductal carcinoma, ovarian cancer, and endometrial cancer), non-small cell lung cancer, pancreatic cancer, thyroid cancer, kidney cancer (e.g., renal cell carcinoma), and bladder cancer (e.g., urothelial cell carcinoma). A cancer can be “a cancer that expresses B7-H4”. Such a term refers to a cancer that contains cells that express B7-H4. The cancer can be a solid tumor that expresses B7-H4. The cancer can be a primary tumor, or it can be an advanced or metastatic cancer.

Unless expressly specified or obvious from the context, as used herein, the term “or” is understood to be inclusive. The term “and/or” as used herein in a phrase such as “A and/or B” is intended to include both “A and B”, “A or B”, “A”, and “B”. Similarly, the term “and/or” as used in a phrase such as “A, B, and/or C” is intended to cover each of the following embodiments: A, B, and C; A, B, or C; A or C; A or B; B or C; A and C; A and B; B and C; A alone; B alone; and C alone.

As used herein, the terms “about” and “approximately” when used to modify a numerical value or numerical range mean that a deviation of 5% to 10% above and a deviation of 5% to 10% below the stated value or range is still within the intended meaning of the stated value or range.

Conventional methods for preparing antibodies include hybridoma technology, recombinant DNA technology, transgenic animal technology, single B cell antibody preparation technology, ribosome display technology, etc. (1) Hybridoma technology. It is a conventional method for isolating monoclonal antibodies, which was first applied by George Kohler and Cesar Milstein in 1975. However, a considerable number of patients show anti-drug antibody reactions to monoclonal antibodies isolated from mouse hybridoma cells. That is, the protein drugs have potential immunogenicity and can induce the human to produce anti-drug antibodies (ADAs). ADAs may reduce the efficacy of drugs, or cause allergic reactions, or even threaten life. In addition, mouse monoclonal antibodies have weak affinity for Fc receptors on the surface of immune cells such as natural killer cells, produce a weak antibody-dependent cell-mediated cytotoxicity, and exhibit low tumor cell-killing ability. Their half-life in human blood circulation is short, and their cytotoxicity lasts for a short time. In addition, the prerequisite for the application of hybridoma technology is that host animals need to be immunized with diseases to induce specific monoclonal antibodies, which is ethically not feasible in human, and in turn leads to the subsequent production of human monoclonal antibodies. (2) Recombinant DNA technology. It can produce chimeric antibodies containing constant region and variable region genes derived from human origin in mice. This chimeric antibody reduces the risk of human anti-mouse antibody response (HAMA) in patients. Humanized antibodies are developed by transplanting mouse-derived CDR regions into human variable region to further reduce the risk of ADA responses against mouse variable region genes in chimeric antibodies. However, humanization can reduce the probability of ADA responses to a greater extent, but it cannot eliminate the probability of ADA responses. 30% of murine origin is still retained, which can induce human anti-mouse responses. In addition, mouse monoclonal antibodies bind poorly to human Fc receptors, which can lead to the loss of a series of indirect protective mechanisms, such as antibody-dependent cell-mediated cytotoxicity, complement-dependent cytotoxicity, phagocytosis, and opsonization. (3) Construction of transgenic mice. It is necessary to produce mouse embryos that lack the original antibody genes through transgenic technology, then human antibody genes are transplanted into mice to cause the mice to express human antibodies, and fully human antibodies are isolated through conventional hybridoma technology. The antibodies obtained in this way are more immune diverse than conventional chimeric antibodies. However, mice need to be cultivated from the embryonic stage, which is more time-consuming and technically complex than other antibody production technologies. In addition, the gene sequence that needs to be implanted is extremely long, making it difficult to ensure accurate expression. (4) Single B cell antibody preparation technology. It is a newly developed in vitro cloning and expression technology for single antigen-specific B cell antibody genes. Different from conventional hybridoma technology, the fusion of lymphocyte B cells and myeloma cells is not required, thereby preventing the challenges in fusion and screening of positive cells accordingly. In this method, the natural pairing between light and heavy chain variable regions is retained, thereby displaying advantages of good gene diversity, high efficiency, full humanization, a small number of cells required, etc. However, the difficulty in adopting this high-throughput technology lies in the fact that it requires very high-end instruments and expertise, such as flow cytometers, next-generation sequencers (NGS), and robotic screening systems for screening cultured memory B cells. Therefore, it is challenging and difficult to adopt this method in countries with limited resources and small biotechnology start-ups because it requires a lot of financial support. In addition, some target antigens are difficult to be prepared, and a large number of suitable human donors are required. (5) Ribosome display technology. Since this technology is a completely in vitro operating system, it does not rely on cell technology and is not restricted by the in vivo environment, and toxic proteins do not affect the growth of host bacteria, which expands the library capacity and molecular diversity of the display library. In addition, conversion between in vitro and in vivo systems is not required in the ribosome display technology. The entire process only involves replication and amplification through PCR technology, which shortens the library construction time and simplifies screening. Mutations can also be introduced through PCR technology to increase molecular diversity, thereby obtaining high-affinity antibodies. However, the biggest defect of the ribosome display technology is that mRNA is easy to be degraded, and the “protein-ribosome-mRNA” ternary complex has poor stability.

Phage display technology was first proposed by George P. Smith in 1985. George P. Smith has proved that foreign genes can be inserted into the filamentous phage p III gene, so that the peptide encoded by the inserted gene can be expressed as a fusion with the protein encoded by the phage p III gene, and the fusion protein can be displayed on the phage surface as the daughter phage is reassembled. Even with foreign peptides, the phage capsid terminal protein III can still maintain its original physiological function, and the phage can survive stably in vitro. At the same time, the foreign protein can still maintain a relatively stable and independent spatial structure and biological activity. According to the fusion, the recombinant peptide can still interact with the target or external ligand. After the phage surface display technology was proposed in 1985, the antibody molecule was the first molecule with natural protein function expressed on the phage surface, so that the establishment of the phage antibody library was developed. The phage antibody library can include different types of antibodies, including human heavy chain antibody fragments, shark or camel antibodies, single chain Fv (scFv, i.e., a single chain antibody), Fab fragments, etc. Ordinary antibodies usually have high affinity for targets, but high immunogenicity. Polypeptides have small molecular weights and are easy to be synthesized, but are easy to be enzymatically hydrolyzed in the systemic circulation and are not suitable for in vivo application. Small molecular compounds, such as folic acid, have small molecular weights and good stability, but are not highly targeted to tumors. ScFv is a small molecule consisted of a heavy chain variable region and a light chain variable region of an antibody linked by a peptide chain, which is the smallest functional structural unit with antibody activity. Due to its small relative molecular mass, weak immunogenicity, and strong penetrability, the scFv can easily penetrate the intercellular space to reach the target cell. In addition, due to the advantages of short manufacturing period, simple operation, and low cost, the phage display technology is used by more and more researchers to efficiently screen and quickly separate tumor-specific antibodies.

### A First Aspect of the Present Disclosure

The present disclosure provides an antibody specifically targeting B7-H4, wherein the antibody includes a heavy chain CDR1 shown in SEQ ID NO: 6, a heavy chain CDR2 shown in SEQ ID NO: 7, and a heavy chain CDR3 shown in SEQ ID NO: 8; and a light chain CDR1 shown in SEQ ID NO: 9, a light chain CDR2 shown in SEQ ID NO: 10, and a light chain CDR3 shown in SEQ ID NO: 11.

In an example, the antibody includes a heavy chain variable region shown in SEQ ID NO: 4.

In an example, the antibody includes a light chain variable region shown in SEQ ID NO: 5.

The type of the antibody is not specifically limited in the present disclosure, which can be, but is not limited to, a monoclonal antibody, wherein the antibody further includes a heavy chain constant region and a light chain constant region. Species from which the heavy chain constant region and the light chain constant region are derived is not specifically limited in the present disclosure, which can be, but is not limited to, any one of human, mouse, rabbit, sheep, cattle, horse, pig, dog, cat, camel, donkey, deer, mink, chicken, duck, and goose.

Types of the heavy chain constant region and the light chain constant region of the antibody is not specifically limited in the present disclosure, which can be, but are not limited to, IgG1, IgG2, IgG3, IgG4, IgA, IgM, IgE, or IgD.

### A Second Aspect of the Present Disclosure

The present disclosure provides a recombinant protein, which includes one or more of the heavy chain CDR1, the heavy chain CDR2, the heavy chain CDR3, the light chain CDR1, the light chain CDR2, the light chain CDR3, the heavy chain variable region, and the light chain variable region defined in the first aspect.

### A Third Aspect of the Present Disclosure

The present disclosure provides a detection reagent, a detection kit, or a medicament, which include the antibody in the first aspect, or the recombinant protein in the second aspect.

### A Fourth Aspect of the Present Disclosure

The present disclosure provides a nucleic acid, which includes a nucleic acid fragment for encoding the antibody in the first aspect or the recombinant protein in the second aspect.

### A Fifth Aspect of the Present Disclosure

The present disclosure provides a recombinant expression vector, which includes the nucleic acid in the fourth aspect.

The type of the recombinant expression vector is not specifically limited in the present disclosure, which can be, but is not limited to, an antibody expression vector. The type of the antibody expression vector is not specifically limited in the present disclosure, which can be, but is not limited to, a bacterial plasmid, a bacteriophage, a yeast plasmid, a plant cell virus, a mammalian cell virus, or a combination thereof. For example, the antibody expression vector is a PCDNA3.1 expression vector.

### A Sixth Aspect of the Present Disclosure

The present disclosure provides a host cell, which includes the nucleic acid in the fourth aspect or any recombinant expression vector in the fifth aspect.

The type of the host cell is not specifically limited in the present disclosure, which includes, but is not limited to, CHO cells, COS cells, NSO cells, HeLa cells, BHK cells, or HEK293 cells.

### A Seventh Aspect of the Present Disclosure

The present disclosure provides a method for preparing the antibody in the first aspect or the recombinant protein in the second aspect, which includes the following steps: culturing the host cell in the sixth aspect, and isolating the antibody or the recombinant protein from the obtained culture.

### An Eighth Aspect of the Present Disclosure

The present disclosure provides use of the antibody in the first aspect or the recombinant protein in the second aspect in the manufacture of a medicament for treating tumors.

It can be understood that the type of the tumor is not specifically limited in the present disclosure, which can be any type of tumor that is positive for B7-H4 expression, such as, colorectal cancer that is positive for B7-H4 expression.

The embodiments of the present disclosure will be described in detail below in conjunction with examples. It should be understood that these examples are only used to illustrate the present disclosure and are not intended to limit the scope of the present disclosure. The experimental methods for which specific conditions are not specified in the following examples are preferably referred to the guidance provided in the present disclosure, and can also be based on the experimental manual or normal conditions in the art, or can also be based on the conditions recommended by the manufacturer, or refer to experimental methods known in the art.

In the following specific examples, the measured parameters of raw material components can have slight deviations within the range of weighing accuracy, unless otherwise specified. The parameters for temperature and time allow acceptable deviations caused by instrument test accuracy or operation accuracy.

In order to make the technical problems solved in the present disclosure, technical solutions, and beneficial effects clearer and more understandable, the present disclosure is further described in detail below in conjunction with the examples. It should be understood that the specific examples described herein are only used to explain the present disclosure and are not used to limit the present disclosure.

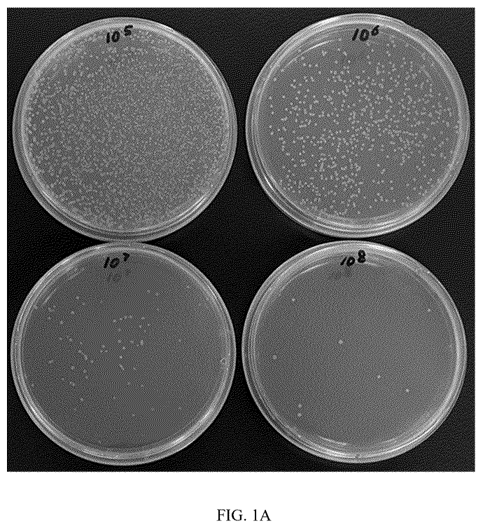

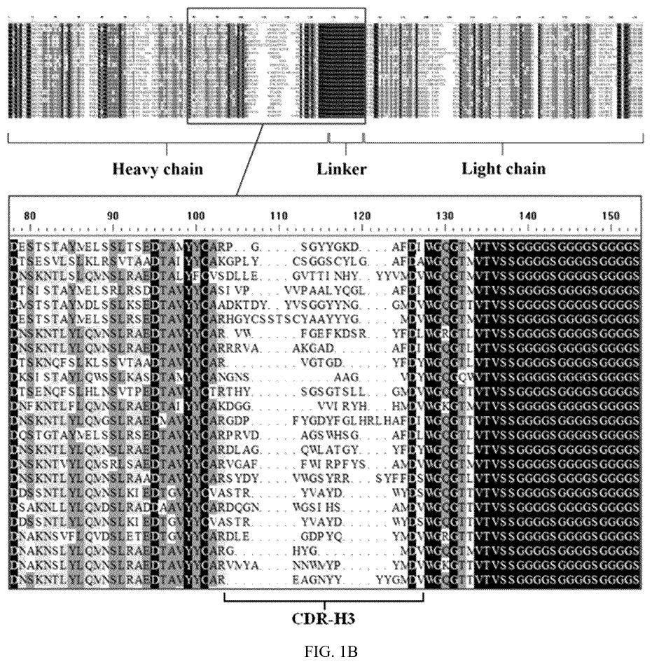

Example 1 Construction of phage display human natural single-chain antibody library
(1) 398 samples of peripheral blood mononuclear cells isolated from healthy adults were collected from the Laboratory Department of the First Affiliated Hospital of Fujian Medical University. The collection process of all peripheral blood samples was approved by the Ethics Committee, all volunteers signed informed consent forms, and all serum samples were anonymized.
(2) An original single-chain antibody library was constructed according to the steps in the literature with DOI: 10.1016/0022-2836 (91) 90498-u.
(3) After the ligation product was transformed into TG1 competent cells by electroporation, the diluted bacterial solution was spread onto a culture plate for culture.
(4) The library capacity was estimated by calculating the number of positive monoclonal clones after dilution on the culture plate.
(5) Monoclonal colonies were randomly selected and sent to Sangon Biotech (Shanghai) Co., Ltd. for DNA sequencing. The diversity of the library was analyzed based on the sequencing results. The positive monoclonal colonies in the culture plate are shown in FIG. 1A. The monoclonal colonies were diluted in a dilution of 1:10⁸, seeded on the culture plate, and cultured overnight at 37° C. in a constant temperature incubator, with eight single bacterial colonies obtained. For a dilution of 1:10⁷, about 76 single bacterial colonies on the culture plate were obtained. The total volume of bacteria solution after electroporation was 8 mL, so that the library capacity was estimated to be about 6.0×10⁹ according to a calculation formula of 76×10⁷×8. The library was packaged by using the helper phage VCSM13 to obtain a phage display human natural single-chain antibody library in a concentration of 1.8×10¹³ cfu/mL. For 24 randomly selected monoclonal antibodies, their DNA sequencing results are shown in FIG. 1B. In the figure, the dark area represents the conserved sequence of the antibody (i.e., the backbone region of the antibody); and the CDR regions of all single-chain antibodies are completely different, especially the CDR3 region of the heavy chain antibody, indicating that it is the most important antigen binding region. The heavy chain CDR-H3 regions of the 24 randomly selected monoclonal antibodies have 7 to 19 amino acids, reflecting the diversity of the hypervariable region of the antibody.

In summary, the constructed library exhibits a high-capacity and broad genetic diversity. An antibody library with high capacity and broad diversity is a critical prerequisite for enhancing screening efficiency and obtaining high-affinity antibodies.

### Example 2 Screening of B7-H4 Single-Chain Antibody

(1) Human B7-H4 protein (Sino Biological, Cat #10738-H08H-100) used in this application was purchased from Sino Biological Inc. (Beijing). The human B7-H4 protein consists of 234 amino acids with a purity of greater than 95%. The amino acid sequence of the human B7-H4 is known in the art, which is also provided herein, containing an IgC extracellular domain and an IgV extracellular domain.

Human B7-H4: >sp|Q7Z7D3|VTCN1_HUMAN| 25-259
OS = Homo sapiens OX = 9606 GN = VTCN1 PE = 1 SV = 1
(SEQ ID NO: 1)
LIIGFGISGRHSITVTTVASAGNIGEDGILSCTFEPDIKLSDIVIQWLKEGVL
GLVHEFKEGKDELSEQDEMFRGRTAVFADQVIVGNASLRLKNVQLTDAGTYKCYII
TSKGKGNANLEYKTGAFSMPEVNVDYNASSETLRCEAPRWFPQPTVVWASQVDQ
GANFSEVSNTSFELNSENVTMKVVSVLYNVTINNTYSCMIENDIAKATGDIKVTESEI
KRRSHLQLLNSKAS

(2) ELISA plate for protein coating was activated under UV irradiation for 30 mins. The human B7-H4 protein was diluted with PBS to be 100 μg/mL, and coated onto wells overnight at 4° C. The next day, the supernatant was discarded, the plate was washed three times with PBS, and the wells were filled with a PBS solution containing 5 wt % skim milk and blocked at room temperature for 2 hours. Next, the blocking solution was discarded, and the plate was washed three times with PBS.

(3) An appropriate amount of phage original library in a titer of 1012 to 1013 TU was added, and incubated at 37° C. for 2 hours. Then, the phage supernatant was removed. The wells were washed several times by filling with a PBST solution (i.e., a PBS solution containing 0.05 wt % Tween-20) and shaking in a micro-oscillator for 5 mins at each time. The number of the washing steps depends on the number of rounds of solid phase affinity screening, in which 5 times in the first round, 10 times in the second round, 13 times in the third round, and 15 times in the fourth round. After that, the wells were washed with PBS 3 times, 5 mins each time.

(4) A 100 μL phage elution solution with pH of 2.2 was added to the wells. The wells were shaken at a high speed in a micro-oscillator for 10 mins. The solution in the wells was completely aspirated, and immediately, a 5 μL phage neutralization solution with pH of 9.1 was added to adjust the pH value of the solution to be near neutral. A bottle of ER2738 bacterial solution in the logarithmic growth phase was prepared. A small amount of eluted phage was aspirated for gradient dilution and titer determination, and the rest of phages were all added to the ER2738 bacterial solution in the logarithmic growth phase for infection and phage amplification.

(5) The titer of the amplified phage was determined according to the method in Example 1. Since the number of eluted phages in different rounds varies greatly, the gradient dilution range is reasonably designed based on the single bacterial colonies obtained in the previous round. The dilution degree for the first round is designed to be in a range from 1:103 to 1:107. The bacterial solution for phage amplification which was infected with phage was spread onto an LB large solid culture medium plate containing 100 μg/mL ampicillin, 20 μg/mL tetracycline, and 1 wt % glucose, and cultured at 30° C. overnight until a bacterial film was formed on the surface of the plate.

(6) The titer of the amplified phage was measured based on the number of the bacterial colonies on the plate after a gradient dilution. 5 to 8 mL of LB liquid culture medium was added to the surface of the large solid culture medium plate, and the bacterial film was gently scraped off. A small amount of the bacterial suspension was taken and added to 100 mL of LB liquid culture medium containing 100 μg/mL ampicillin, 20 μg/mL tetracycline, and 1 wt % glucose, with an initial OD600 of less than or equal to 0.1. The remaining bacterial suspension was stored as glycerol bacteria at −80° C.

(7) The bacterial solution was cultured at 37° C. with shaking at 250 rpm until the logarithmic phase. Helper phage VCS-M13 was added at an infection ratio of bacteria to helper phage of 1:20 based on 5×10⁸ bacterial cells per OD600 unit, incubated at room temperature for 30 mins, and centrifuged at 3300 g and 4° C. for 10 mins. The bacterial pellet was resuspended in 500 mL LB liquid culture medium containing 100 μg mL ampicillin and 25 μg/mL kanamycin, and cultured at 30° C. overnight.

(8) The bacteria solution after overnight culture was centrifuged at 10,800 g for mins, and the supernatant was discarded. 20% volume of PEG/NaCl solution (containing 20 wt % PEG and 2.5 M NaCl) was added, mixed well, and left to stand at 4° C. for at least 1 h. Then, the solution was centrifuged at 10,800 g for 10 mins, and the phage pellet was resuspended in 10 mL PBS. The phage suspension was washed by centrifuging at 13,000 g for 10 mins to remove bacterial debris. The supernatant was collected for gradient dilution and titer determination. The dilution degree is designed to be in a range from 1:109 to 1:1013 for the first round. The amplified phage was diluted to a working solution concentration ranging from 1012 TU/mL to 1013 TU/mL for the next round of affinity screening.

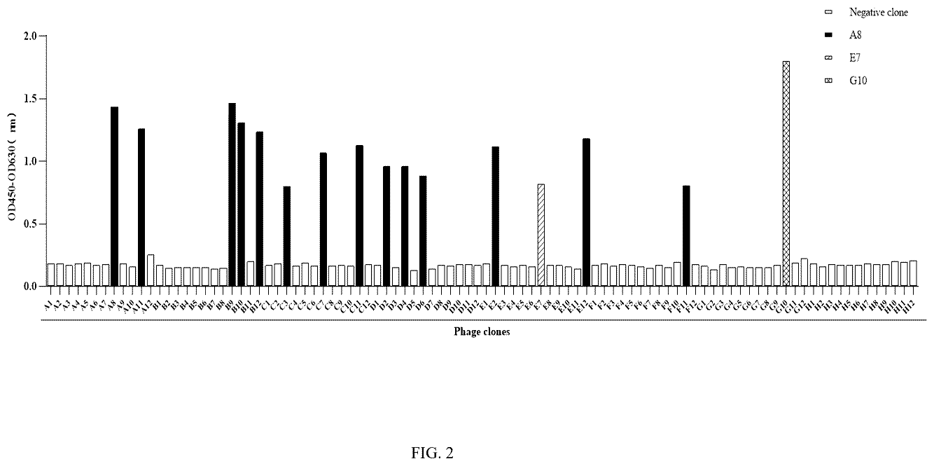

(9) As shown in FIG. 2, steps (1) to (7) were repeated for a total of 4 rounds of affinity screening. The titers of input and output phages were measured in each round, and the results are shown in Table 1.

TABLE 1
Titer of NpSc21 in four rounds of screening

| | | | | Enrichment |
|---|---|---|---|---|
| | | | Recovery ratio | factor (ratio n/ |
| Round | Input (tu) | Output (tu) | (output/input) | ratio n − 1) |
| 1 | 2.00 × 10¹² | 5.00 × 10⁴ | 2.78 × 10⁻⁸ | — |
| 2 | 2.00 × 10¹² | 4.40 × 10⁶ | 2.20 × 10⁻⁶ | 79.14 |
| 3 | 2.00 × 10¹² | 9.70 × 10⁷ | 4.85 × 10⁻⁵ | 22.05 |
| 4 | 2.00 × 10¹² | 8.80 × 10⁸ | 4.40 × 10⁻⁴ | 9.07 |

### Example 3 Preparation of Phage Soluble Antibody Fragments and ELISA Detection

(1) On the first day, approximately 10⁵ of the phages were taken from the eluted phages after the third and fourth rounds of screening, and infected 200 μL of HB2151 bacteria in the logarithmic growth phase. The bacteria solution was incubated in a water bath at 37° C. for 30 mins, and centrifuged at 6000 rpm and 4° C. for 10 mins. The bacteria pellet was diluted in 10 folds. Then, the diluted bacteria solution was spread onto SOB plates containing 100 μg/mL ampicillin, 2 wt % glucose, and 100 μg/mL nalidixic acid at dilutions of 1:10, 1:102, and 1:103, and then cultured at 30° C. overnight.

(2) On the second day, single colonies were randomly selected, and placed in a 96-deep-well bacterial culture plate with wells each added with 500 μL of 2×YT medium containing 100 μg/mL ampicillin and 2 wt % glucose, and then cultured at 250 rpm/min and 30° C. in a shaker overnight.

(3) On the third day, a disposable 96-well plate was taken, 50 μL of 80 wt % glycerol was added to each well, and then 150 μL of bacterial solution was added to each well. The plate was sealed with a sealing film, and stored at −80° C.

(4) At the same time, another 96-well bacterial culture plate was taken, with 500 μL of 2×YT medium containing 100 μg/mL ampicillin and 2 wt % glucose already added to each well. 5 μL of bacterial solution was taken from each well of the 96-deep-well bacterial culture plate in step (2), and added to each well of the new 96-well bacterial culture plate in step (4), then incubated at 37° C. with shaking in a shaker until reaching the logarithmic phase.

(5) The 96-well bacterial culture plates in step (3) and step (4) were centrifuged at 3000 rpm for 20 mins by using a plate centrifuge. The supernatant was carefully removed. The plates were turned upside down, and the remaining supernatant was absorbed by sterilized toilet paper. In this step, when discarding the supernatant, the plate cannot be tapped to avoid pouring out the bacterial pellet. Next, 500 μL of 2×YT medium containing 100 μg/mL ampicillin and 1 mM IPTG was added to each well of the two 96-deep-well bacterial culture plates, and cultured at 30° C. and 250 rpm in a shaker for 16 to 24 hours.

(6) A 96-well ELISA plate was placed in a clean bench, and activated under UV irradiation for more than 30 mins. The antigen protein solution to be coated was diluted with PBS buffer to a concentration ranging from 1 μg/mL to 10 μg/mL, and added to per well in 100 μL. The plate was sealed with a sealing film, and incubated at 4° C. overnight.

(7) On the fourth day, the protein solution in the 96-well ELISA plate in step (6) was recovered, which can be directly discarded if the protein is not expensive. The plate was washed with PBS (200 μL/well) three times, 5 minutes each time. After each washing, the plate was shaken and tapped on the table to remove the solution inside as much as possible. The plate was then blocked with PBS containing 5 wt % skim milk at 37° C. for 2 hours, 300 μL per well.

(8) After blocking, the ELISA plate in step (7) was washed with PBS (200 μL per well) three times, 5 minutes each time. The two 96-deep-well plates incubated overnight in step (5) were centrifuged at 3000 rpm and 4° C. for 20 mins, and 150 μL of supernatant was taken from each well and added to the wells of the ELISA plate. The ELISA plate was then sealed with a sealing film, and incubated at 4° C. overnight.

(9) On the fifth day, the supernatant in the ELISA plate was discarded, and the ELISA plate was washed with a PBST solution (i.e., a PBS solution containing 0.1 wt % Tween 20) using 200 μL per well three times, 5 mins each time. The anti-HA-tag antibody was diluted with PBS containing 2 wt % skim milk, at a dilution ratio specified by the antibody instruction. 100 μL of the diluted antibody solution was added to each well of the plate, and incubated at 37° C. for 2 hours (or incubated at 4° C. overnight).

(10) The antibody solution in the plate in step (9) was discarded. The plate was washed three times with PBST (PBS containing 0.1 wt % Tween 20) using 200 μL per well, 5 mins each time. Then, the plate was washed three times with PBS using 200 μL per well, 5 mins each time. A secondary antibody with HRP label (the species of the secondary antibody is determined by the primary antibody) was diluted with PBS containing 2 wt % skim milk, at a dilution ratio specified by the antibody instruction. 100 μL of the diluted secondary antibody solution was added to each well of the plate, and incubated at 37° C. for 1.5 hours.

(11) The secondary antibody solution in the plate was discarded. The plate was washed three times with PBST (PBS containing 0.1 wt % Tween 20) using 200 μL per well, 5 mins each time. Then, the plate was washed three times with PBS using 200 μL per well, 5 mins each time.

(12) 100 μL of color development solution was added to each well, and incubated at room temperature in the dark for 10 to 20 mins. The color development was observed every 2 to 3 mins.

(13) 50 μL of dilute sulfuric acid was added to each well to terminate the reaction.

(14) OD630 and OD450 were measured. The final detection result was obtained by subtracting the OD630 value from the OD450 value. The results are shown in FIG. 2. A total of 16 positive monoclonal clones (i.e., Npsc-A8, All, B9, B10, B12, C3, C7, C11, D2, D4, D6, E2, E7, E12, F11, and G10) are obtained.

(15) 16 positive NpSc monoclonal clones with relatively high absorbance values were selected from the ELISA results, which were cultured for expansion, and then were stored in glycerol or sequenced. The sequencing primers are shown in SEQ ID NO:2 and SEQ ID NO:3. The sequencing results showed that three different scFv sequences were obtained from the 16 positive monoclonal clones. Among them, 14 clones had exactly the same scFv sequence, and one of the monoclonal clones, Npsc-A8, was selected for subsequent experiments; and the other two scFv sequences were respectively from monoclonal clones, Npsc-E7 and Npsc-G10.

TABLE 2
Sequencing primers for Npsc21

| Name of primers | Sequence (5′→3′) |
|---|---|
| Npsc-21 sequencing | AAGACAGCTATCGCGATTGCAG |
| primer (SEQ ID NO: 2) | |
| Npsc-21 sequencing | GCCCCCTTATTAGCGTTTGCCATC |
| primer (SEQ ID NO: 3) | |

### Example 4 Identification of Specificity of Single-Chain Antibody Candidates

### 4.1 Binding Activity of Single-Chain Antibody Candidates to B7-H4 Positive Cells

(1) Resuspension of cells: After trypsin digestion, cells were centrifuged, the supernatant was discarded, and the cells were resuspended in PBS containing 1% (v/v) FBS and counted to adjust the concentration to 2×10⁶ cells/mL.
(2) Setting controls: For a blank control, an isotype control, and samples to be tested, 100 μL of the resuspended cells in step (1) was added to each tube, so that each tube contains 2×10⁵ cells.
(3) Incubation with primary antibody: A blank control tube did not contain any antibody. A negative control tube was added with 50 μL of negative single-chain antibody, which was the monoclonal single-chain antibody that was negative in ELISA in Example 3. Tubes to be tested were respectively added with 50 μL of antibody candidates (i.e., Npsc-A8, Npsc-E7, Npsc-G10). The mixture in tubes was fully mixed, and incubated at 4° C. for 30 mins. During the incubation, the tubes were shaken every 10 mins to allow the cells to be fully reacted with the antibodies.
(4) Washing of cells: An appropriate amount of PBS containing 1% (v/v) FBS was added to each tube. The tubes were centrifuged at 2000 rpm for 3 mins, and the supernatant was discarded.
(5) Resuspension of cells: The cells in each tube were resuspended in 100 μL of PBS containing 1% FBS.
(6) Incubation with secondary antibody: An appropriate amount of fluorescently labeled secondary antibody was added to the isotype control tube and the test tubes, mixed thoroughly, and incubated at 4° C. in the dark for 30 mins. During the incubation, the tubes were shaken every 10 mins.
(7) Washing of cells: 2 mL PBS was added to each tube, the tubes were centrifuged at 2000 rpm for 3 mins, and the supernatant was discarded. This process was conducted twice.
(8) On-machine testing: Cells in each tube were resuspended in 200 μL PBS. The blank tube and the isotype control tube were tested first, and then the test tubes were tested.

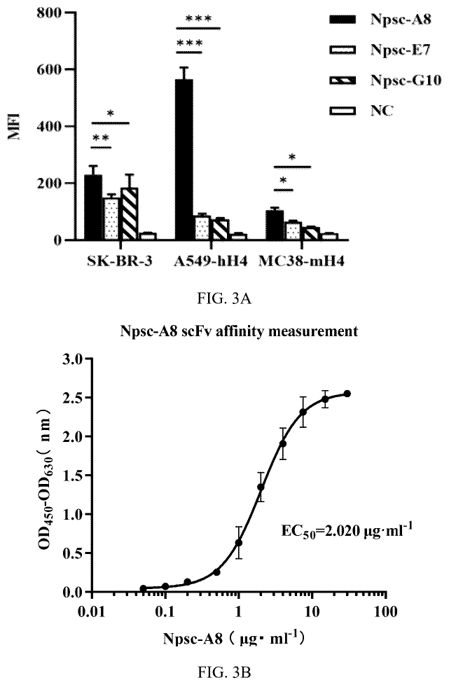

FIG. 3A shows the flow cytometry results of the three single-chain antibody candidates against SK-BR-3 (i.e., human breast adenocarcinoma cells endogenously expressing B7-H4), A549-hH4 (i.e., human alveolar basal epithelial adenocarcinoma cells overexpressing B7-H4), and MC38-mH4 (i.e., mouse colon cancer cells overexpressing B7-H4). Npsc-E7 and Npsc-G10 single-chain antibodies weakly bind to cells expressing B7-H4. In contrast, the binding activity of Npsc-A8 single-chain antibody to cells expressing B7-H4 is stronger than that of Npsc-E7 and Npsc-G10 single-chain antibodies. Therefore, the Npsc-A8 single-chain antibody was selected as the preferred monoclonal antibody for subsequent experiments. The Npsc-A8 single-chain antibody includes sequences shown in SEQ ID NO: 4 and SEQ ID NO: 5. After analyzing by using http://bioinf.org.uk/abs/, its CDR regions are shown in SEQ ID NOs: 6-11.

SEQ ID NO: 4. Heavy chain amino acid sequence of Npsc-A8 single-chain antibody

QVQLQESGGGLMQVGGSLRLSCTASGFVFGDYGMNWVRQAPGKGLE
WVSLIDTYGASTFYADSVKGRFTISRDNSKQTVFLQMDGLRADDTAMYF
CAKPILMFPAFDLWGPGTMVTVSS

SEQ ID NO: 5. Light chain amino acid sequence of Npsc-A8 single-chain antibody

DVVMTQSPSSLSASVGDRVTITCRASQGIRNDLAWYQKKPGKAPKRLIY
AASSLQSGVPSRFSGSGYGAEFSLTISSLQPEDFATYFCLHHSQYPWTF
GQGTKVDIKR

SEQ ID NO: 6. CDR1 amino acid sequence of heavy chain variable domain

GFVFGDYGMN

SEQ ID NO: 7. CDR2 amino acid sequence of heavy chain variable domain

ASTFYADSVKGRFTISRDN

SEQ ID NO: 8. CDR3 amino acid sequence of heavy chain variable domain

PILMFPAFDL

SEQ ID NO: 9. CDR1 amino acid sequence of light chain variable domain

RASQGIRNDLA

SEQ ID NO: 10. CDR2 amino acid sequence of light chain variable domain

AASSLQS

SEQ ID NO: 11. CDR3 amino acid sequence of light chain variable domain

LHHSQYPWT

4.2 Measurement of Affinity of the Npsc-A8 scFv
(1) ELISA plate was placed in a clean bench, and activated under UV irradiation for more than 30 mins. Human B7-H4 protein (Sinobiological, a catalog number: 10738-HO8H-100) was coated onto wells of the ELISA plate overnight at 4° C. at 2 μg/mL. The Npsc-A8 single-chain antibody was diluted to 20 μg/mL, 10 μg/mL, 7.5 μg/mL, 5 μg/mL, 2.5 μg/mL, 1 μg/mL, 0.5 μg/mL, 0.25 μg/mL, 0.1 μg/mL, and 0.05 μg/mL, which were respectively added to wells in 100 μL per well.
(2) The remaining steps were conducted according to steps (7) to (14) in Example 3.
(3) The OD450 nm value was measured. A standard curve was plotted, and the EC₅₀ value was obtained.

The ELISA test results of the affinity of the Npsc-A8 single-chain antibody are shown in FIG. 3B, and the concentration for 50% of maximal effect (EC₅₀) is 2.020 μg/mL.

### Example 5 Preparation of Full-Length Antibody of the Preferred Monoclonal Antibody

### 5.1 Construction of Expression Vector

(1) The Npsc-A8 single-chain antibody has a weak affinity. In order to improve the affinity of the Npsc-A8 single-chain antibody, the Npsc-A8 single-chain antibody was modified into a full-length antibody. The Npsc-A8 scFv gene was amplified by PCR method. The light chain variable region gene and the heavy chain variable region gene were respectively introduced into the expression vector PCDNA3.1 containing the human IgG1 light chain constant region, IgG1 heavy chain constant region, and signal peptide. 50 μL of the PCR reaction system was shown in Table 3, and the PCR reaction conditions were 98° C. for 10 sec, 71° C. for 5 sec, 72° C. for 5 sec, a total of 35 cycles, and stored at 4° C. The final product was identified through 1 wt % agarose gel electrophoresis. For the expression vector for expressing heavy chain, NheI and Kpn I were selected as upstream and downstream restriction sites for enzyme digestion. For the expression vector for expressing light chain, NheI and BamHI were selected as upstream and downstream restriction sites for enzyme digestion. After enzyme ligation, the ligation products were chemically transformed into DH5a competent cells. The primers were designed by using Snapgene software, in which the forward primer of heavy chain (H-F), the reverse primer of heavy chain (H-R), the forward primer of light chain (L-F), the reverse primer of light chain (L-R) were as follows.

H-F (SEQ ID NO: 12):
CTATCGATTGAATTCCACCATGGACTGGACCTGGAGCAT
H-R (SEQ ID NO: 13):
CAAGCTTGGGAGCGGCCGCTCATTTACCCGGAGACAG
L-F (SEQ ID NO: 14):
CTATCGATTGAATTCCACCATGGACATGAGGGTCCCCGC
L-R (SEQ ID NO: 15):
CAAGCTTGGGAGCGGCCGCTTAACACTCTCCCCTGTT

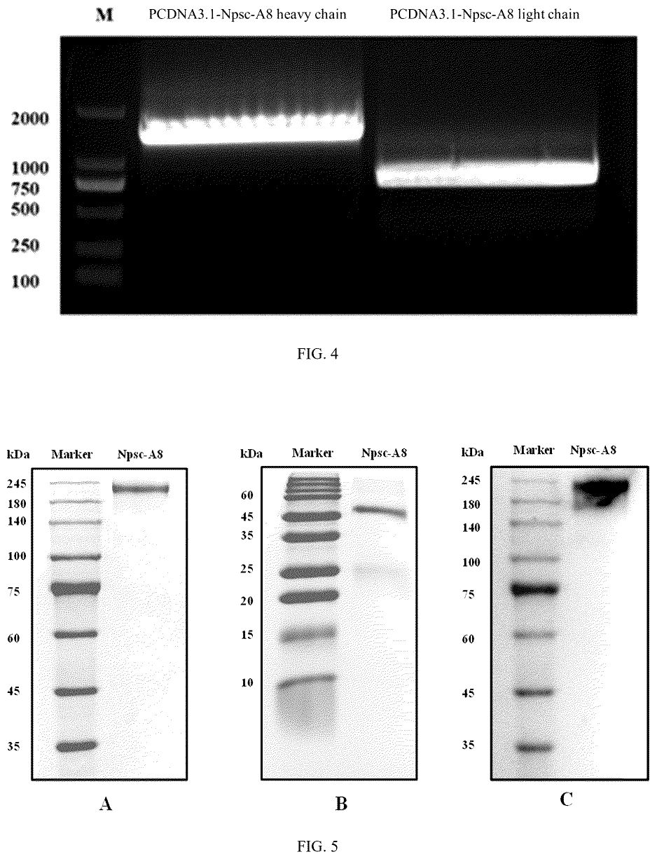

The PCR amplification results are shown in FIG. 4. 746 bp of light chain variable region and constant region genes of the Npsc-A8 antibody, and 1439 bp of heavy chain variable region and constant region genes of the Npsc-A8 antibody were obtained. After the transformation, the single clones on the plate were sent to Sangon Biotech (Shanghai) Co., Ltd. for DNA sequencing. The clones which have the correct sequences after verification by sequencing were subjected to large-scale plasmid extraction by using the NucleoBond® Xtra Maxi Plus EF kit from MACHEREY-NAGEL.

TABLE 3
PCR reaction system for construction of expression vectors
for expressing light and heavy chains of Npsc-A8 antibody

| Reagent | Amount | |
|---|---|---|
| PrimeSTAR Max Premix (2×) | 25 | μL |
| H-F/L-F | 2 | μL |
| H-R/L-R | 2 | μL |
| DNA | 1 | μL |

| | |
|---|---|
| ddH₂O | Up to 50 μL |

### 5.2 Expression and Purification of the Npsc-A8 Full-Length Antibody

### (1) Recovery and Culture of HEK293T Cell

1) HEK293T cells frozen in a liquid nitrogen container were taken into a tube. The tube containing the cells was placed in a water bath at 37° C. with shaking to quickly thaw the cells within 1 to 2 minutes. The water in the water bath cannot be higher than the mouth of the tube. The tube wall was wiped to dry, and wiped with 75% (v/v) alcohol. The cells were added to a 15 mL centrifuge tube, 5 mL of preheated DMEM culture medium was added, and the tube was centrifuged at 1000 rpm for 5 mins.
2) The supernatant was discarded. The cells were resuspended in DMEM medium. A certain amount of the cell suspension was taken, diluted n times, counted to adjust the density to be 2 to 5×10⁴ cells/cm², and cultured in an incubator at 37° C. and 5% CO₂.
3) After culturing for 24 hours, the cells were observed, and sub-cultured when the cells reached 80% to 90% confluency.

### (2) Transfection

1) Splitting of cells: HEK293T cells were digested with trypsin, collected, and counted. 1×10⁷ cells were seeded onto a 15 cm cell culture dish, and a total of 10 cell culture dishes were set. The cell culture dishes were incubated in an incubator at 37° C. and 5% CO₂ for 8 hours to 24 hours. Cells were transfected when they were fully attached to the wall, specifically, when the attached cells occupied 80% to 90% of the total area of the culture dish.
2) Preparation of a PEI-DNA mixture: One of the culture dishes was operated as follows. Two 1.5 mL EP tubes were taken. Plasmids, preferably in a total amount of 30 μg, including 15 μg of heavy chain-PCDNA3.1 plasmids and 15 μg of light chain-PCDNA3.1 plasmids, were added into one of the EP tubes, and then diluted to a total volume of 500 μL with OPTI MEM medium, and mixed well. In the other EP tube, 100 μM PEI stock solution was diluted to a 10 μM PEI solution with OPTI MEM medium, and mixed well. Then, 500 μL of the 10 μM PEI solution was added to the 500 μL plasmid solution diluted with OPTI MEM medium, mixed well, and left to stand at room temperature for 10 mins to 20 mins. Finally, the 1 mL PEI-DNA mixture was added dropwise to the cell culture medium containing monolayer cells in step 1). The culture dish was gently shaken to mix well, and incubated in an incubator at 37° C. and 5% CO₂. The remaining nine culture dishes were operated in the same way.
3) After culturing for 8 hours, the culture medium in the culture dishes was replaced with DMEM culture medium containing 1% (v/v) serum and valproic acid (a final concentration of 4 μM) preheated at 37° C., and further cultured for about 5 days. The cells were observed. The supernatant of the culture medium was collected, and centrifuged at 4000 rpm for 30 mins to remove cells and cell debris. The supernatant was filtered with a 0.22 μm filter head. The filtrate was collected for purification.

### (3) Purification of Antibody

1) A chromatography column and a filtration system were connected, and the flow rate was set to 1 mL/min.
2) The chromatography column was washed with 15 mL elution buffer with pH of 2.0.
3) The chromatography column was equilibrated with 25 mL PBS.
4) The filtrate obtained in step 3) was loaded by using a suction pipe.
5) After sample loading, the culture medium in the column was eluted with 25 mL of PBS.
6) Ten new 1.5 mL EP tubes were labeled, 100 L 1 M Tris Buffer (pH 9.0) was added to each tube.
7) The target antibody protein bound on the chromatography column was eluted with 8 to 10 mL elution buffer (pH 3.5) loaded with a 10 mL syringe, and collected into EP tubes, with 1 mL of the target antibody protein solution collected in each tube.
8) The concentration of the target antibody protein solution was measured by Nanodrop instrument.
9) The collected target antibody protein solution was analyzed by SDS-PAGE and Western-blot. The results are shown in FIG. 5. FIGS. 5A and 5B show the results of SDS-PAGE electrophoresis of the purified Npsc-A8 full-length antibody under non-reducing and reducing conditions, by using Coomassie-stained gels. The results showed that the Npsc-A8 full-length antibody has a molecular weight of about 180 kDa under the non-reducing condition, and the antibody has two subunits with molecular weights of 25 kDa and 55 kDa under the reducing condition, which are consistent with the theoretical value of the sequence of the antibody. The result of Western blot also showed a dark band at a molecular weight of about 180 kDa, see FIG. 5C.

### Example 6 Evaluation of Specificity of the Npsc-A8 Full-Length Antibody

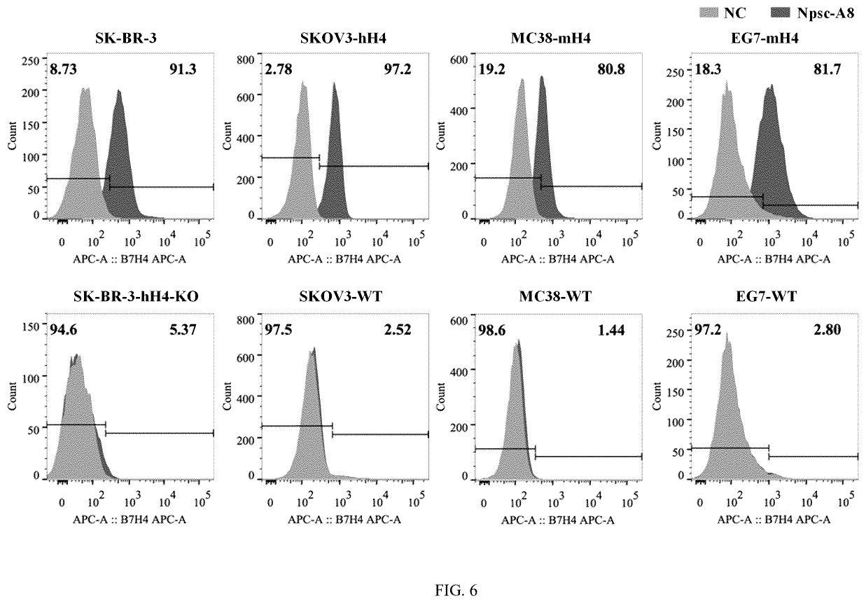

The specificity of the Npsc-A8 full-length antibody was identified according to the method in 4.1 of Example 4. The results in FIG. 6 show that the Npsc-A8 full-length antibody binds to endogenous B7-H4 on SK-BR-3 cells and overexpressed B7-H4 on SKOV3-hH4, MC38-mH4, and E.G7-OVA-mH4. In contrast, it shows no binding to B7-H4-knockout SK-BR-3-hH4-KO cells or wild-type controls lacking B7-H4 expression (SKOV3-WT, MC38-WT, and E.G7-OVA-WT). In general, at the same concentration, the Npsc-A8 full-length antibody binds to human tumor cells at an affinity higher than that to mouse tumor cells.

### Example 7 Binding Site Analysis of the Npsc-A8 Full-Length Antibody

(1) A 96-well ELISA plate was placed in a clean bench, and activated under UV irradiation for more than 30 mins. The human B7-H4 protein (Sinobiological, a catalog number: 10738-HO8H-100), mouse B7-H4 protein (from a laboratory of Fujian Medical University), mouse B7-H4 IgV-like domain protein (from a laboratory of Fujian Medical University), and mouse B7-H4 IgC-like domain protein (from a laboratory of Fujian Medical University) were respectively coated onto wells of the 96-well ELISA plate at a concentration of 2 μg/mL, and incubated at 4° C. overnight. The Npsc-A8 full-length antibody protein obtained in Example 5 was diluted to 10 μg/mL with PBS buffer, and added to wells in 100 μL per well.
(2) The remaining steps were conducted according to steps (6) to (13) in Example 3. 6H3 and 8H4 were used as control antibodies. 6H3 is a mouse anti-human B7-H4 IgC domain antibody, referring to patent application publication number CN 104955475 A. 8H4 is a hamster anti-human B7-H4 IgV domain antibody, referring to DOI: 10.1016/s1074-7613 (03) 00152-3.
(3) OD450 nm value was measured.

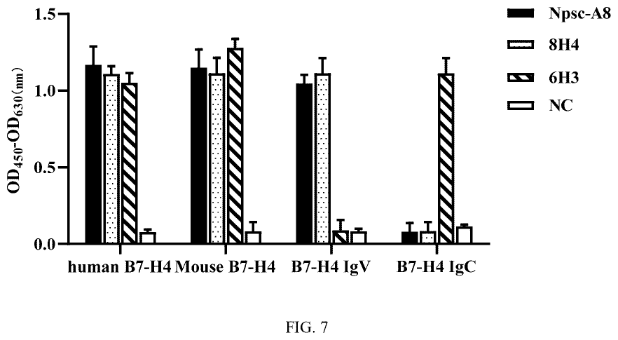

The results are shown in FIG. 7. The Npsc-A8 full-length antibody binds to human B7-H4 protein and mouse B7-H4 protein. The Npsc-A8 full-length antibody binds to mouse B7-H4 IgV-like domain protein, but it does not bind to mouse B7-H4 IgC-like domain protein.

### Example 8 Effect of the Npsc-A8 Full-Length Antibody on Inhibition of T Cell Proliferation by B7-H4

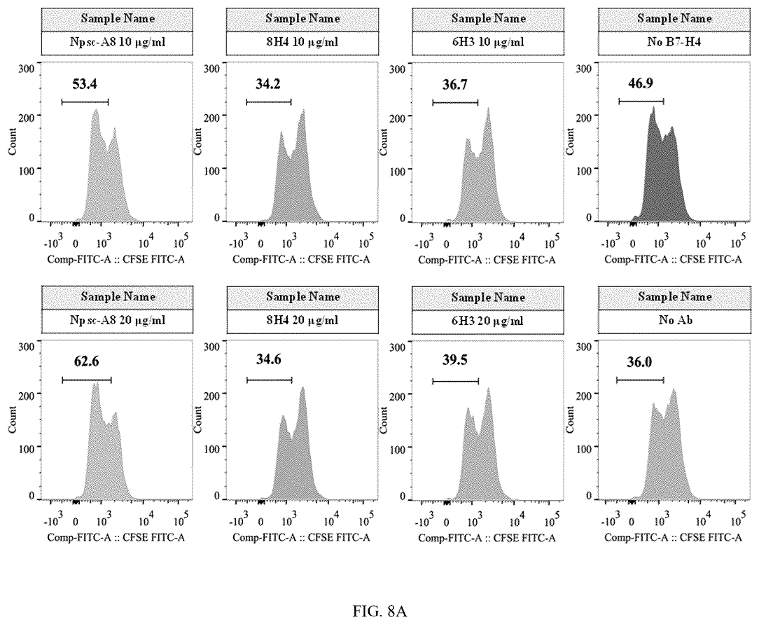

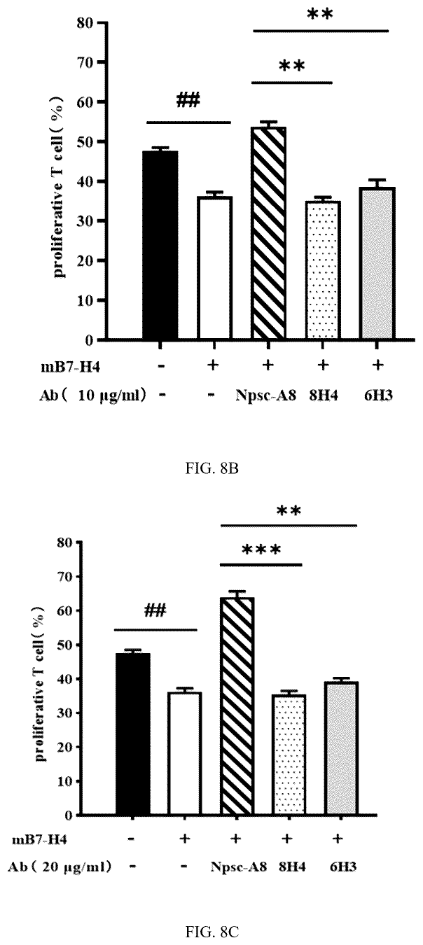

(1) Mouse B7-H4 protein (20 μg/mL, from a laboratory of Fujian Medical University) and Anti-CD3 (1 μg/mL) were respectively coated onto a 96-well cell culture plate, and incubated at 4° C. overnight.
(2) On the next day, the 96-well cell culture plate was washed with PBS to remove unbound B7-H4 protein and unbound Anti-CD3, and then blocked with 3% BSA at 37° C. for 2 hours.
(3) The blocking solution was discarded. The Npsc-A8 full-length antibody, 8H4 antibody, and 6H3 antibody at concentrations of 10 μg/mL and 20 μg/mL, and Anti-CD28 (1 μg/mL) were respectively added to wells of the 96-well cell culture plate, and incubated at 37° C. for 1 hour.
(4) T cell isolation and pre-staining with CFSE
1) Inguinal lymph nodes from wild-type mice were isolated aseptically.
2) The lymph nodes were ground by rough surface of a glass slide, and filtered with a 70 μm cell sieve.
3) The filtrate was centrifuged at 500 g for 5 mins. The cells were resuspended in PBS to 1×10⁷ cells/mL, CFSE at a final concentration of 3 μM was added, and the mixture was incubated at 37° C. for 8 mins.
4) 10 mL of pre-cooled PBS containing 1% (v/v) serum was added. The mixture was incubated at 4° C. for 10 mins, centrifuged at 500 g for 5 mins, and the supernatant was discarded.
5) The lymphocytes were resuspended in RPMI-1640 medium containing 10 IU/mL IL-2, and adjusted to a concentration of 2×10⁶ cells/mL. 2×10⁵ T cells were added to each well, and incubated for three days. The proliferation of T cells was observed under a microscope.
(5) In vitro proliferation of the T cells mediated by the Npsc-A8 full-length antibody was detected by flow cytometry. The results are shown in FIG. 8. The CFSE proliferation peak (i.e., a secondary peak) can well reflect the proliferation of the T cells. The more T cells proliferate, the greater the proportion of the secondary peak to the original peak. As shown in FIG. 8, the proliferation of T cells in the group with mB7-H4 protein but without any antibody was significantly lower than that in the group without B7-H4, indicating the inhibition of T cell proliferation by B7-H4. Among the Npsc-A8 full-length antibody group, 6H3 antibody group, and 8H4 antibody group, the proportion of proliferation peak is highest in the Npsc-A8 full-length antibody group, indicating the most significant T cell proliferation. The results show that the Npsc-A8 full-length antibody can effectively reverse the inhibition of T cell proliferation by B7-H4, and restore and enhance the proliferation ability of T cells. In contrast, the 6H3 antibody and the 8H4 antibody do not have the efficacy.

### Example 9 Efficacy of the Npsc-A8 Full-Length Antibody on Killing B7-H4 Positive Tumor Cells by T Cells

### 9.1 Isolation of Mouse Spleen T Cells

1) An OT-I/Rag1 mouse was killed by CO₂ asphyxiation. A small cut was made in its abdomen, and torn open slowly. The mouse spleen was placed into a 6 cm small culture dish filled with 2 to 3 mL washing buffer (i.e., RPMI-1640 complete medium: PBS=1:9).
2) The spleen was divided into small parts, and sequentially ground by the rough surface of a glass slide.
3) The cells in the small culture dish were transferred into a 50 mL centrifuge tube. The remaining cells in the slide and the small culture dish were collected by rinsing with the washing buffer, and a final volume of about 30 mL spleen cell solution was obtained.
4) The spleen cell solution was centrifuged at 500 g for 5 mins, the supernatant was discarded, and the cells were flicked to disperse the cell pellets.
5) 3 mL of red blood cell lysis buffer was added, gently pipetted to uniformly mix the spleen cells, and left to stand for 1 to 2 minutes.
6) 30 mL washing buffer was added to terminate the lysis reaction.
7) The cell mixture was filtered with a cell sieve, and the filtrate was collected into a fresh 50 mL centrifuge tube.
8) The filtrate was centrifuged at 500 g/min for 5 mins, the supernatant was discarded, and the cells were flicked to disperse the cell pellets.
9) 30 mL washing buffer solution was added, and the cell solution was filtered into a fresh 50 mL centrifuge tube.
10) The filtrate was centrifuged at 500 g/min for 5 mins, the supernatant was discarded, and the cells were flicked to disperse the cell pellets.
11) The cells were resuspended in 6 mL RPMI-1640 complete medium, and counted.
12) According to the cell counting result, the cell density was adjusted to 1×10⁷ cells/mL. 0.3 μL of IL-2 (an original concentration of IL-2 was 1000 IU/mL, and a final concentration was 100 IU/mL) was added to promote the proliferation of OT-I cells.
   9.2 Labeling of Target Cells (i.e., Tumor Cells) with CFSE
1) The suspensions of mouse T lymphoma cells (E.G7-OVA-WT) and mouse T lymphoma cells that overexpress B7-H4 (E.G7-OVA-mH4) in the logarithmic growth phase as the target cells were centrifuged. Next, the cells were resuspended in PBS, and counted.
2) The two cell suspensions were respectively added to 15 mL centrifuge tubes, and each tube contained 2×10⁵ cells. The tubes were centrifuged at 1500 rpm for 3 mins.
3) The cells were resuspended in 1 mL PBS, 0.6 μL CFSE (an original concentration was 5 mM, and a final concentration was 3 μM) was added, and the mixture was incubated at 37° C. for 8 mins.
4) 10 mL of pre-cooled PBS containing 1% (v/v) serum was added, and the mixture was left to stand at 4° C. for 10 mins.
5) The tubes were centrifuged at 800 g for 5 mins, and the supernatant was discarded.
6) The cells were resuspended in 10 mL of PBS containing 1% serum, and washed by centrifugation.
7) The cells were resuspended in 1 mL of RPMI-1640 complete medium, counted, and diluted to a concentration of 1×10⁵ cells/mL. 1×10⁴ target cells (100 μL) were contained in each well.

### 9.3 Co-Incubation of Target Cells and T Cells

1) 1×10⁶ OT-I cells were co-cultured with the target cells at a ratio of T cells to target cells of 100:1 for 24 hours, and cell death was evaluated.
2) After 24 hours of co-incubation, the mixed cells in each well were labelled with a death marker with near-IR wavelength of 633 nm to 635 nm, 0.02 μL per tube, and left to stand at room temperature for 8 to 10 mins (or at 4° C. for 30 mins).
3) 1 mL of PBS was added to each tube, and the tubes were centrifuged at 500 g for 5 mins.
4) The cells were detected by flow cytometry.

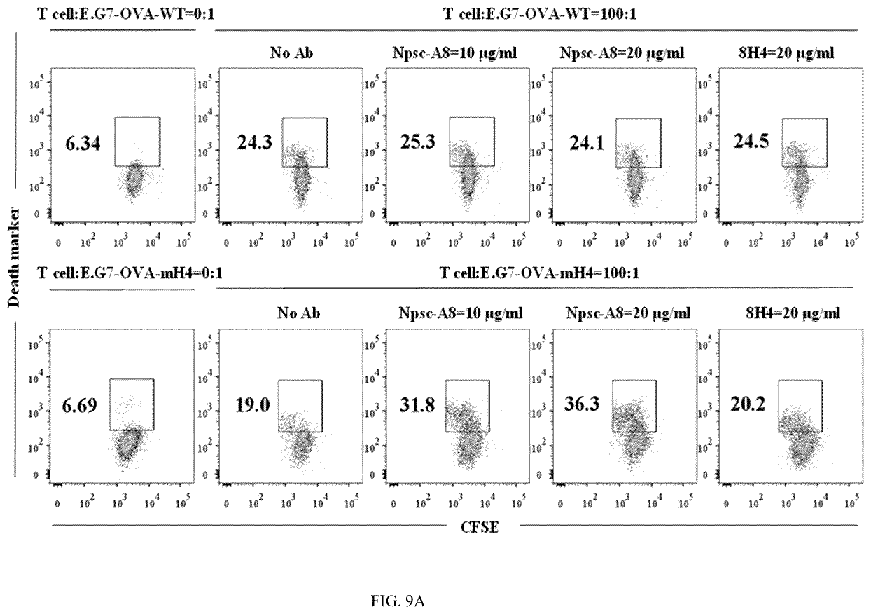

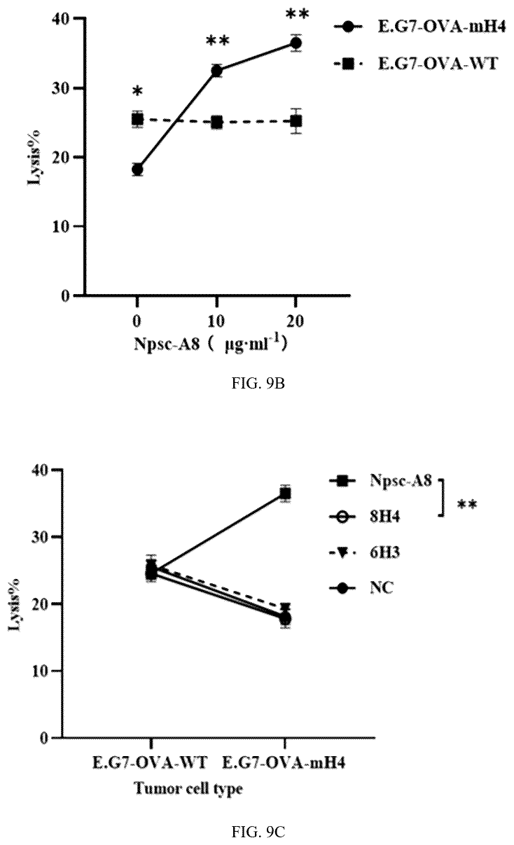

The results are shown in FIG. 9. In the absence of antibodies, T cells exhibited stronger cytotoxic activity against E.G7-OVA-WT cells than the E.G7-OVA-mH4 cells, due to the immunosuppression by B7-H4. However, after adding the Npsc-A8 full-length antibody, the cytotoxic activity of T cells on the E.G7-OVA-mH4 cells was enhanced, and was in dose-dependent relation, which indicates that the immunosuppressive effect of B7-H4 on T cells can be effectively reversed by the Npsc-A8 full-length antibody. In contrast, the 8H4 and 6H3 antibodies do not have the efficacy.

### Example 10 Therapeutic Efficacy of Anti-B7-H4 Blocking Antibody

(1) Construction of MC38-mH4 subcutaneous tumor model: MC38-mH4 cells in the logarithmic growth phase were prepared, and inoculated subcutaneously on the right flank of 8-week-old C57BL/6 female mice at 0.8×10⁶/100 μL/mice, with 5 mice in each group.

The MC38-mH4 mouse colon cancer tumor model was used to test the blocking effect of the Npsc-A8 full-length antibody on B7-H4 function. MC38-mH4 cells were provided by the Institute of Immunotherapy, Fujian Medical University. The MC38-mH4 cells were cultured in RPMI-1640 medium containing 10% (v/v) fetal bovine serum, 1.5 g/L sodium bicarbonate, 4.5 g/L glucose, 10 mM HEPES, and 1.0 mM sodium pyruvate.

1.2×10⁷ MC38-mH4 cells resuspended in 1.5 mL PBS were injected subcutaneously into 8-week-old wild-type C57/BL6 female mice. In order to test the therapeutic efficacy of anti-B7-H4 blocking antibodies, on day 5, 1 mg/kg of Npsc-A8 full-length antibody and 1 mg/kg of 6H3 antibody as a control anti-B7-H4 antibody were respectively intraperitoneally injected in the MC38-mH4 model.
(2) Starting from day 5, tumors of the mice were measured every three days, recorded as an average diameter of tumor (i.e., (long diameter+short diameter)/2). The growth curve was plotted.
(3) When showing average diameter of tumor greater than 15 mm, or symptoms such as weight loss, hunchback, cessation of eating, inability to walk, and lethargy, the mice were sacrificed by CO₂ anesthesia. The survival period of the mice was recorded, and a survival curve was drawn.
(4) Evaluation of the therapeutic efficacy of the Npsc-A8 full-length antibody in the MC38-mH4 tumor-bearing model

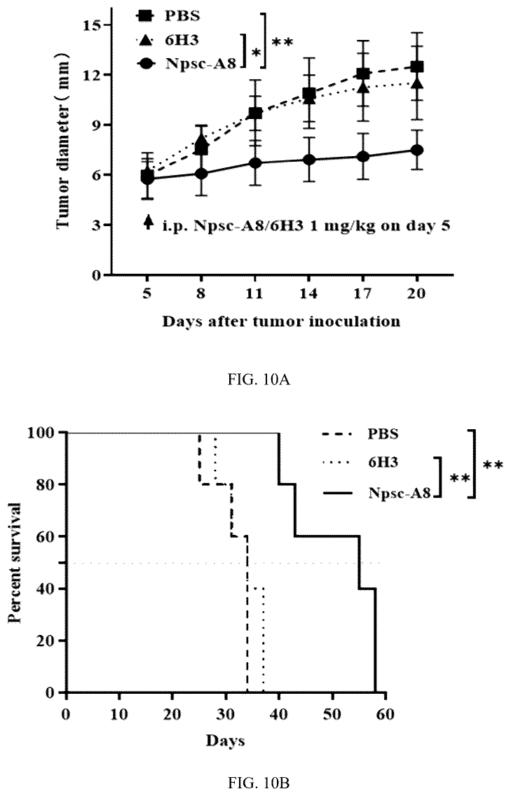

The results are shown in FIG. 10A. Compared to the 6H3 antibody monotherapy group and the PBS control group, the Npsc-A8 full-length antibody monotherapy had a significant anti-tumor effect (P<0.05) (n=5). The survival curves of mice after different drug treatments are shown in FIG. 10B. Mice in the PBS and 6H3 antibody monotherapy groups exhibited significantly shorter survival than those in the Npsc-A8 full-length antibody monotherapy group (P<0.01)

The technical features of the above-mentioned embodiments can be combined arbitrarily. In order to make the description concise, not all possible combinations of the technical features are described in the embodiments. However, as long as there is no contradiction in the combination of these technical features, the combinations should be considered as in the scope of the present disclosure.

The above-described embodiments are only several implementations of the present disclosure, and the descriptions are relatively specific and detailed, but they should not be construed as limiting the scope of the present disclosure. It should be understood by those of ordinary skill in the art that various modifications and improvements can be made without departing from the concept of the present disclosure, and all fall within the protection scope of the present disclosure.

Therefore, the patent protection of the present disclosure shall be defined by the appended claims.

1. An antibody specifically targeting B7-H4 or antigen-binding fragment thereof, comprising:
a heavy chain CDR1 shown in SEQ ID NO: 6, a heavy chain CDR2 shown in SEQ ID NO: 7, and a heavy chain CDR3 shown in SEQ ID NO: 8; and
a light chain CDR1 shown in SEQ ID NO: 9, a light chain CDR2 shown in SEQ ID NO: 10, and a light chain CDR3 shown in SEQ ID NO: 11.

2. The antibody specifically targeting B7-H4 or antigen-binding fragment thereof according to claim 1, wherein:
the antibody comprises a heavy chain variable region shown in SEQ ID NO: 4.

3. The antibody specifically targeting B7-H4 or antigen-binding fragment thereof according to claim 1, wherein the antibody further comprises a heavy chain constant region and a light chain constant region; and
the antibody comprises constant regions derived from any one of IgG1, IgG2, IgG3, IgG4, IgA, IgM, IgE, or IgD.

4. A recombinant protein comprising the antibody specifically targeting B7-H4 or antigen-binding fragment thereof according to claim lone or more of the heavy chain CDR1, the heavy chain CDR2, the heavy chain CDR3, the light chain CDR1, the light chain CDR2 and the light chain CDR3 defined in claim 1, and the heavy chain variable region and the light chain variable region defined in claim 2.

5. A medicament, comprising the antibody specifically targeting B7-H4 or antigen-binding fragment thereof according to claim 1.

6. A nucleic acid comprising a nucleic acid fragment for encoding the antibody specifically targeting B7-H4 or antigen-binding fragment thereof according to claim 1.

7. A recombinant expression vector comprising the nucleic acid according to claim 6.

8. A host cell comprising the nucleic acid according to claim 6.

9. A method for preparing the antibody specifically targeting B7-H4 or antigen-binding fragment thereof according to claim 1, comprising:
culturing a host cell, wherein the host cell comprises a nucleic acid encoding the antibody specifically targeting B7-H4 or antigen-binding fragment thereof according to claim 1, and
isolating the antibody or antigen-binding fragment thereof from the obtained culture.

10. (canceled)

11. The antibody specifically targeting B7-H4 or antigen-binding fragment thereof according to claim 1, wherein the antibody comprises a light chain variable region shown in SEQ ID NO: 5.

12. The antibody specifically targeting B7-H4 or antigen-binding fragment thereof according to claim 3, wherein the heavy chain constant region and the light chain constant region are each independently derived from any species selected from the group consisting of human, mouse, rabbit, sheep, cow, horse, pig, dog, cat, camel, donkey, deer, mink, chicken, duck, and goose.

13. The recombinant expression vector according to claim 7, wherein the recombinant expression vector is an antibody expression vector.

14. The recombinant expression vector according to claim 13, wherein the recombinant expression vector is selected from the group consisting of a bacterial plasmid, a bacteriophage, a yeast plasmid, a plant cell virus, a mammalian cell virus, and a combination thereof.

15. The host cell according to claim 8, wherein the host cell is a CHO cell, a COS cell, an NSO cell, a HeLa cell, a BHK cell, or a HEK293 cell.

16. A method for treating a tumor in a subject in need thereof, comprising administering to the subject an effective amount of the antibody specifically targeting B7-H4 or antigen-binding fragment thereof according to claim 1.

17. The method according to claim 16, wherein the tumor is any type of tumor that is positive for B7-H4 expression.

18. The method according to claim 17, wherein the tumor is a colorectal cancer that is positive for B7-H4 expression.


SEQUENCE LISTING (15 sequences)

SEQ ID NO: 1 - protein, 235 aa, synthetic construct
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKAS

SEQ ID NO: 2 - DNA, 22 bp, synthetic construct
  source 1..22: mol_type = other DNA
aagacagctatcgcgattgcag

SEQ ID NO: 3 - DNA, 24 bp, synthetic construct
  source 1..24: mol_type = other DNA
gcccccttattagcgtttgccatc

SEQ ID NO: 4 - protein, 119 aa, synthetic construct
QVQLQESGGGLMQVGGSLRLSCTASGFVFGDYGMNWVRQAPGKGLEWVSLIDTYGASTFYADSVKGRFTISRDNSKQTVFLQMDGLRADDTAMYFCAKPILMFPAFDLWGPGTMVTVSS

SEQ ID NO: 5 - protein, 108 aa, synthetic construct
DVVMTQSPSSLSASVGDRVTITCRASQGIRNDLAWYQKKPGKAPKRLIYAASSLQSGVPSRFSGSGYGAEFSLTISSLQPEDFATYFCLHHSQYPWTFGQGTKVDIKR

SEQ ID NO: 6 - protein, 10 aa, synthetic construct
GFVFGDYGMN

SEQ ID NO: 7 - protein, 19 aa, synthetic construct
ASTFYADSVKGRFTISRDN

SEQ ID NO: 8 - protein, 10 aa, synthetic construct
PILMFPAFDL

SEQ ID NO: 9 - protein, 11 aa, synthetic construct
RASQGIRNDLA

SEQ ID NO: 10 - protein, 7 aa, synthetic construct
AASSLQS

SEQ ID NO: 11 - protein, 9 aa, synthetic construct
LHHSQYPWT

SEQ ID NO: 12 - DNA, 39 bp, synthetic construct
  source 1..39: mol_type = other DNA
ctatcgattgaattccaccatggactggacctggagcat

SEQ ID NO: 13 - DNA, 37 bp, synthetic construct
  source 1..37: mol_type = other DNA
caagcttgggagcggccgctcatttacccggagacag

SEQ ID NO: 14 - DNA, 39 bp, synthetic construct
  source 1..39: mol_type = other DNA
ctatcgattgaattccaccatggacatgagggtccccgc

SEQ ID NO: 15 - DNA, 37 bp, synthetic construct
  source 1..37: mol_type = other DNA
caagcttgggagcggccgcttaacactctcccctgtt